SEQUENCE LISTING (83 sequences)

SEQ ID NO: 1 - protein, 169 aa, Mus musculus
MGRRFLVTVRIQRAGRPLQERVFLVKFVRSRRPRTASCALAFVNMLLRLERILRRGPHRNPGPGDDDGQRSRSSSSAQLRCRFELRGPHYLLPPGARRSAGRLPGHAGGAARVRGSAGCARCLGSPAARLGPRAGTSRHRAIFAFRWVLFVFRWVVFVYRWERRPDRRA

SEQ ID NO: 2 - protein, 19 aa, Artificial Sequence
  Synthetic
KFVRSRRPRTASCALAFVN

SEQ ID NO: 3 - protein, 30 aa, Artificial Sequence
  Synthetic
KFVRSRRPRTASCALAFVNMLLRLERILRR

SEQ ID NO: 4 - protein, 9 aa, Artificial Sequence
  Synthetic
XXXXXXXXX

SEQ ID NO: 5 - protein, 13 aa, Human immunodeficiency virus
MGYGRKKRRQRRR

SEQ ID NO: 6 - protein, 28 aa, Artificial Sequence
  Synthetic
XXXXXXXXXKFVRSRRPRTASCALAFVN

SEQ ID NO: 7 - protein, 39 aa, Artificial Sequence
  Synthetic
XXXXXXXXXKFVRSRRPRTASCALAFVNMLLRLERILRR

SEQ ID NO: 8 - RNA, 21 nt, Artificial Sequence
  Synthetic
caacaggagucuaaucaaguu

SEQ ID NO: 9 - RNA, 21 nt, Artificial Sequence
  Synthetic
ggaccacuuucccuacuuuuu

SEQ ID NO: 10 - RNA, 21 nt, Artificial Sequence
  Synthetic
guagugggcccaacaaauuuu

SEQ ID NO: 11 - RNA, 21 nt, Artificial Sequence
  Synthetic
gcugggaucaagauuauuauu

SEQ ID NO: 12 - DNA, 21 bp, Artificial Sequence
  Synthetic
cagucgcguuugcgacuggtt

SEQ ID NO: 13 - protein, 4 aa, Artificial Sequence
  Synthetic
IEAL

SEQ ID NO: 14 - DNA, 18 bp, Artificial Sequence
  Synthetic
gtttttgtttgtttgttt

SEQ ID NO: 15 - DNA, 20 bp, Artificial Sequence
  Synthetic
acacccactcctccaccttt

SEQ ID NO: 16 - DNA, 20 bp, Artificial Sequence
  Synthetic
ttcctcttgtgctcttgctg

SEQ ID NO: 17 - DNA, 20 bp, Artificial Sequence
  Synthetic
gcaggctgcactatcaacaa

SEQ ID NO: 18 - DNA, 20 bp, Artificial Sequence
  Synthetic
tcgaaggctcctcaacctta

SEQ ID NO: 19 - DNA, 22 bp, Artificial Sequence
  Synthetic
aaagtctaccaccgaatcccta

SEQ ID NO: 20 - DNA, 22 bp, Artificial Sequence
  Synthetic
ccaaaacacaaaaccaaaatga

SEQ ID NO: 21 - DNA, 22 bp, Artificial Sequence
  Synthetic
gaatggaggaatcttggcgttc

SEQ ID NO: 22 - DNA, 23 bp, Artificial Sequence
  Synthetic
tctttggtttcttgggtagtggg

SEQ ID NO: 23 - DNA, 23 bp, Artificial Sequence
  Synthetic
tgtagaggatgaggcgtgttgag

SEQ ID NO: 24 - DNA, 22 bp, Artificial Sequence
  Synthetic
agcaagtgggtggactattcgg

SEQ ID NO: 25 - DNA, 22 bp, Artificial Sequence
  Synthetic
cacgaaaagggctgaaatgttc

SEQ ID NO: 26 - DNA, 24 bp, Artificial Sequence
  Synthetic
ggtgtttgtaagaggtggtatcgc

SEQ ID NO: 27 - DNA, 24 bp, Artificial Sequence
  Synthetic
gccagtgtcctttactttccctcc

SEQ ID NO: 28 - DNA, 22 bp, Artificial Sequence
  Synthetic
ttcagtttctccccttaggccc

SEQ ID NO: 29 - DNA, 22 bp, Artificial Sequence
  Synthetic
caaatgtgcttgccttttagcc

SEQ ID NO: 30 - DNA, 23 bp, Artificial Sequence
  Synthetic
tgggattacagatgtgagccacc

SEQ ID NO: 31 - DNA, 19 bp, Artificial Sequence
  Synthetic
cacggtcagaccaatttct

SEQ ID NO: 32 - DNA, 20 bp, Artificial Sequence
  Synthetic
tgataggggaggaagagcaa

SEQ ID NO: 33 - DNA, 24 bp, Artificial Sequence
  Synthetic
gctatccaactcctgggaagattc

SEQ ID NO: 34 - DNA, 22 bp, Artificial Sequence
  Synthetic
caatgtctccttgatgggggtc

SEQ ID NO: 35 - DNA, 20 bp, Artificial Sequence
  Synthetic
gaggaaagagcaccttcagc

SEQ ID NO: 36 - DNA, 20 bp, Artificial Sequence
  Synthetic
aggcaatgtctccttgatgg

SEQ ID NO: 37 - DNA, 20 bp, Artificial Sequence
  Synthetic
ccgaaaccggaagatgtcta

SEQ ID NO: 38 - DNA, 20 bp, Artificial Sequence
  Synthetic
agatgtggctcagggatgac

SEQ ID NO: 39 - DNA, 20 bp, Artificial Sequence
  Synthetic
acatcgacctcaccaagagg

SEQ ID NO: 40 - DNA, 20 bp, Artificial Sequence
  Synthetic
tcccatccttcacatccttc

SEQ ID NO: 41 - DNA, 24 bp, Artificial Sequence
  Synthetic
tgataaccttgaacctgcttgtcc

SEQ ID NO: 42 - DNA, 24 bp, Artificial Sequence
  Synthetic
ggctgcttgtgtctctgtatttgc

SEQ ID NO: 43 - DNA, 20 bp, Artificial Sequence
  Synthetic
aaggcggcatacggaaagac

SEQ ID NO: 44 - DNA, 22 bp, Artificial Sequence
  Synthetic
atccaacaaggcactgaccatc

SEQ ID NO: 45 - DNA, 22 bp, Artificial Sequence
  Synthetic
actcacagcgaggatgacaagg

SEQ ID NO: 46 - DNA, 21 bp, Artificial Sequence
  Synthetic
taacgatgccgatgccaatag

SEQ ID NO: 47 - DNA, 22 bp, Artificial Sequence
  Synthetic
ttcagtctcaacgatgccctgg

SEQ ID NO: 48 - DNA, 24 bp, Artificial Sequence
  Synthetic
attagtctcggacaccactctgcc

SEQ ID NO: 49 - DNA, 20 bp, Artificial Sequence
  Synthetic
atcattgcccctccagaacg

SEQ ID NO: 50 - DNA, 22 bp, Artificial Sequence
  Synthetic
gcttcgtcgtattcctgtttgc

SEQ ID NO: 51 - DNA, 22 bp, Artificial Sequence
  Synthetic
aatgtgcctgagaggtccaatg

SEQ ID NO: 52 - DNA, 24 bp, Artificial Sequence
  Synthetic
cgagaaatagggttgtccttcaag

SEQ ID NO: 53 - DNA, 20 bp, Artificial Sequence
  Synthetic
gcaggctgcactatcaacaa

SEQ ID NO: 54 - DNA, 20 bp, Artificial Sequence
  Synthetic
tcgaaggctcctcaacctta

SEQ ID NO: 55 - DNA, 22 bp, Artificial Sequence
  Synthetic
tgtcagaccaccacaaccagac

SEQ ID NO: 56 - DNA, 24 bp, Artificial Sequence
  Synthetic
tggatgccttccttcttcatagtc

SEQ ID NO: 57 - DNA, 20 bp, Artificial Sequence
  Synthetic
acacccactcctccaccttt

SEQ ID NO: 58 - DNA, 20 bp, Artificial Sequence
  Synthetic
ttcctcttgtgctcttgctg

SEQ ID NO: 59 - DNA, 20 bp, Artificial Sequence
  Synthetic
ttatcggtgggatagtttgc

SEQ ID NO: 60 - DNA, 20 bp, Artificial Sequence
  Synthetic
aaccgctaacccgaaaatac

SEQ ID NO: 61 - DNA, 23 bp, Artificial Sequence
  Synthetic
cttgtaatagttgaagcgtgttt

SEQ ID NO: 62 - DNA, 20 bp, Artificial Sequence
  Synthetic
atacctttaactaaaacgtc

SEQ ID NO: 63 - DNA, 25 bp, Artificial Sequence
  Synthetic
tggtgatggacgaggtttagtaagt

SEQ ID NO: 64 - DNA, 26 bp, Artificial Sequence
  Synthetic
aaccaataaaacctactcctccctta

SEQ ID NO: 65 - DNA, 22 bp, Artificial Sequence
  Synthetic
ctgacgctgttcgttctggaga

SEQ ID NO: 66 - DNA, 18 bp, Artificial Sequence
  Synthetic
aagatttgcctccgaacc

SEQ ID NO: 67 - DNA, 22 bp, Artificial Sequence
  Synthetic
acgcctcctcctcctcctctac

SEQ ID NO: 68 - DNA, 20 bp, Artificial Sequence
  Synthetic
agcacacctccgacagccag

SEQ ID NO: 69 - DNA, 23 bp, Artificial Sequence
  Synthetic
gtcacactcggattcctctctcc

SEQ ID NO: 70 - DNA, 23 bp, Artificial Sequence
  Synthetic
ccccaaaaaaaagcagcagacac

SEQ ID NO: 71 - DNA, 19 bp, Artificial Sequence
  Synthetic
caagtgggctcaggagaaa

SEQ ID NO: 72 - DNA, 19 bp, Artificial Sequence
  Synthetic
gtgtgagggtcgtcgtgtt

SEQ ID NO: 73 - DNA, 24 bp, Artificial Sequence
  Synthetic
ttcagaacttactttcagggacgg

SEQ ID NO: 74 - DNA, 22 bp, Artificial Sequence
  Synthetic
aatgctgccaggagagggagtg

SEQ ID NO: 75 - DNA, 22 bp, Artificial Sequence
  Synthetic
tctcatccctcactgttgccac

SEQ ID NO: 76 - DNA, 22 bp, Artificial Sequence
  Synthetic
tgtcattgtcacctctttcccg

SEQ ID NO: 77 - DNA, 20 bp, Artificial Sequence
  Synthetic
ttttacggggcaactacggc

SEQ ID NO: 78 - DNA, 22 bp, Artificial Sequence
  Synthetic
cagtggcatccattagcaggtc

SEQ ID NO: 79 - protein, 17 aa, Artificial Sequence
  Synthetic
XXXXXXXXXSCALAFVN

SEQ ID NO: 80 - DNA, 82 bp, Artificial Sequence
  Synthetic
gatcgaacttggagtccgattgtattaccgtgtgtcaagagcacacggtaatacaatcggactccaagttttttttgaagct

SEQ ID NO: 81 - DNA, 82 bp, Artificial Sequence
  Synthetic
gatcgaagaacacgagtgtaatatagatgaggtgtcaagagcacctcatctatattacactcgtgttcttttttttgaagct

SEQ ID NO: 82 - DNA, 82 bp, Artificial Sequence
  Synthetic
gatcgcctgtgctcttgaagttgtaatggctacgtcaagagcgtagccattacaacttcaagagcacaggttttttgaagct

SEQ ID NO: 83 - DNA, 82 bp, Artificial Sequence
  Synthetic
gatcgccttacggattcctggaggtaacatctattcaagagatagatgttacctccaggaatccgtaaggttttttgaagct

(19) United States
(12) Patent Application Publication
(10) Pub. No.: US 2013/0142784 A1
(43) Pub. Date: Jun. 6, 2013

(54) METHOD OF TREATING TUMOR RESISTANT TO HERCEPTIN OR PACLITAXEL USING FOXM1 INHIBITORS AND DETECTING SAME

(71) Applicants: Pradip Raychaudhuri, Oak Park, IL (US); Janai Carr, Chicago, IL (US)

(72) Inventors: Pradip Raychaudhuri, Oak Park, IL (US); Janai Carr, Chicago, IL (US)

(73) Assignee: The Board of Trustees of the University of Illinois, Urbana, IL (US)

(21) Appl. No.: 13/640,245

(22) PCT Filed: Apr. 7, 2011
(86) PCT No.: PCT/US11/31599

§ 371 Date: Feb. 19, 2013

Related U.S. Application Data
(60) Provisional application No. 61/321,586, filed on Apr. 7, 2010.

(51) Int. Cl. A61K 39/395; A61K 38/17; A61K 31/713; A61K 31/337

(52) U.S. Cl. CPC A61K 39/3955; A61K 31/337; A61K 38/1764; A61K 31/713
USPC 424/133.1; 514/449; 514/19.3; 514/44 A; 424/94.4; 514/315; 514/188; 514/19.9

(57) ABSTRACT

The invention provides methods of treating cancer, especially breast cancer, and in particular HER2/ErbB2 positive breast cancer using a FoxM1 inhibitor in conjunction with trastuzumab and/or paclitaxel. Pharmaceutical compositions comprising a FoxM1 inhibitor in the presence of trastuzumab and/or paclitaxel are also provided. The invention further provides methods of identifying and treating trastuzumab resistant and/or paclitaxel resistant cancer. Also provided are methods of promoting breast tumor cell differentiation.

This invention relates to and claims the benefit of priority from U.S. Provisional Application Ser. No. 61/321,586, filed on Apr. 7, 2010, the disclosure of which is incorporated herein by reference in its entirety.

This invention was made with government support under grant numbers R01 CA124488 and F31 CA136183 awarded by the National Institute of Health. The government has certain rights in the invention.

### BACKGROUND OF THE INVENTION

Breast cancer is the most common female malignancy in most industrialized countries, as it is estimated to affect about 10% of the female population during their lifespan. Although its mortality has not increased along with its incidence, due to earlier diagnosis and improved treatment, it is still one of the predominant causes of death in women.

The mammary gland is a dynamic organ that undergoes continuous cycles of proliferation, differentiation, and apoptosis. During puberty, the rudimentary mammary gland invades the surrounding fat pad and undergoes extensive growth resulting in ductal expansion and formation of a mature branched mammary structure. In early pregnancy, the gland undergoes further growth and tertiary branching to create alveoli or bud-like structures to support milk production. Throughout pregnancy, the epithelium continues to proliferate. After weaning, widespread apoptosis and angiogenic remodeling result in reestablishment of the mature gland (Hennighausen and Robinson, 2005, “Information networks in the mammary gland,” Nat Rev Mol Cell Biol 6:715-25). Thus, dysregulation of proliferation, differentiation and apoptosis in the breast tissue can lead to uncontrolled growth and cancer.

Management of breast cancer currently relies on a combination of early diagnosis and aggressive treatment, which can include one or more treatments such as surgery, radiation therapy, chemotherapy, and hormone therapy. HERCEPTIN (trastuzumab) was developed as a targeted therapy for HER2/ErbB2 positive breast cancer cells, often used in conjunction with other therapies, including the mitotic inhibitor paclitaxel (sold under the trade name TAXOL).

HER2/ErbB2 (also known as HER2, neu, CD340 and p185) stands for human epidermal growth factor receptor 2, encoded by the ERBB2 gene. It is a cell surface receptor tyrosine kinase with no known ligand and functions by forming heterodimers with other family members to promote intracellular signaling (Le et al., 2005, “HER2-targeting antibodies modulate the cyclin-dependent kinase inhibitor p27Kip1 via multiple signaling pathways,” Cell Cycle 4: 87-95). Heterodimerized HER2/ErbB2 normally is involved in signal transduction pathways that include numerous components, such as those in the AKT/PI3K pathway, many of which are also involved in cancer formation and other diseases. Breast tumors with amplified HER2/ErbB2 are characterized by aggressive growth and poor prognosis, which leave patients with few treatment options. HERCEPTIN (trastuzumab) functions to disrupt the interaction between HER2/ErbB2 and its binding partners (Junttila et al., 2009, “Ligand-independent HER2/HER3/PI3K complex is disrupted by trastuzumab and is effectively inhibited by the PI3K inhibitor GDC-0941” Cancer Cell 15: 429-40). However, the mechanisms of the action of trastuzumab are not fully understood (Valabrega et al., 2007, Annals Oncology 18:977-984).

The efficacy of HERCEPTIN as a monotherapy is estimated to be less than 30%; combinatorial treatment with microtubule stabilizing drugs such as paclitaxel increases efficacy to approximately 60% (Burris, HA, 3rd., 2000, “Docetaxel (Taxotere) in HER-2-positive patients and in combination with trastuzumab (HERCEPTIN)” Semin Oncol 27: 19-23). Treatment with HERCEPTIN results in accumulation of the Cdk inhibitor p27 and subsequent G1/S cell cycle arrest, and paclitaxel stalls the entry of mitosis which can lead to cell death. In spite of great promise, however, high doses of HERCEPTIN or paclitaxel result in undesirable side effects. Further, the cancer often develops resistance to HERCEPTIN and/or paclitaxel.

Paclitaxel is used in the treatment of multiple tumor types and has shown particular success in treatment of metastatic breast cancer. Insensitivity to paclitaxel has been shown in cells that overexpress HER2/ErbB2; on average, cells with HER2/ErbB2 amplification require a 100-fold higher dose of paclitaxel to produce the same effect. (Azambuja et al., 2008, “HER-2 overexpression/amplification and its interaction with taxane-based therapy in breast cancer,” Ann Oncol 19: 223-32). Resistance to paclitaxel has also been seen in other non-breast tumors.

Resistance to HERCEPTIN develops quickly and is thought to stem from compensated signaling by other EGF family members or dysregulation of downstream pathways such as PI3K/Akt (Nahta et al., 2004, “P27(kip1) down-regulation is associated with trastuzumab resistance in breast cancer cells,” Cancer Res 64: 3981-6; Pohlmann et al., 2009, “Resistance to Trastuzumab in Breast Cancer,” Clin Cancer Res 15: 7479-7491). HER2/ErbB2 functions upstream of several cell cycle regulating proteins, among which is the oncogenic transcription factor FoxM1. Overexpression or silencing of HER2/ErbB2 directly correlates with FoxM1 levels in mammary cell lines and in transgenic mice (Francis et al., 2009, “FoxM1 is a downstream target and marker of HER2 overexpression in breast cancer” Int J Oncol 35: 57-68; Bektas et al., 2008, “Tight correlation between expression of the Forkhead transcription factor FOXM1 and HER2 in human breast cancer” BMC Cancer 8:42).

FoxM1 is overexpressed not only in breast tumors but also in a broad range of tumor types, including those of neural, gastrointestinal, and reproductive origin (see Bektas et al., supra; Nakamura et al., 2004, “Genome-wide cDNA microarray analysis of gene expression profiles in pancreatic cancers using populations of tumor cells and normal ductal epithelial cells selected for purity by laser microdissection” Oncogene 23: 2385-400; Pilarsky et al., 2004, “Identification and validation of commonly over-expressed genes in solid tumors by comparison of microarray data,” Neoplasia 6: 744-50; Liu et al., 2006, “FoxM1B is overexpressed in human glioblastomas and critically regulates the tumorigenicity of glioma cells,” Cancer Res 66: 3593-602). This expression pattern of FoxM1 is attributed to the ability of FoxM1 to transactivate genes required for cell cycle progression (Wang et al., 2002, “The Forkhead Box m1b transcription factor is essential for hepatocyte DNA replication and mitosis during mouse liver regeneration,” Proc Natl Acad Sci USA 99: 16881-6; Leung et al., 2001, “Over-expression of FoxM1 stimulates cyclin B1 expression,” FEBS Lett 507: 59-66). Increased nuclear staining of FoxM1B found in human basal cell carcinomas suggests that FoxM1 is required for cellular proliferation in human cancers (Teh et al., 2002, Cancer Res. 62: 4773-80). The detailed role of FoxM1 in establishing or facilitating tumor progression and disease management has not been fully elucidated, however.

While significant advances in breast cancer treatment have been made, side effects and both inherent and acquired resistance to existing treatments leave an unmet need for better cancer treatment.

### SUMMARY OF THE INVENTION

Provided herein are compositions and pharmaceutical compositions and methods for therapeutic treatment of breast cancer. Specifically, the invention provides methods for treating breast cancer by administering to a patient a pharmaceutical composition of a FoxM1 inhibitor together with HERCEPTIN (trastuzumab) or paclitaxel. The invention further provides methods for promoting breast tumor cell differentiation by inhibiting FoxM1 activity or expression.

As set forth herein, pharmaceutical compositions in a therapeutically effective amount are provided for inhibiting tumor growth comprising a combination of a FoxM1 inhibitor and either trastuzumab or paclitaxel, wherein the combination is in a therapeutically effective amount, and a pharmaceutically acceptable excipient, diluent or carrier. In certain particular embodiments the pharmaceutical composition comprises a FoxM1 inhibitor and trastuzumab. In certain other embodiments the pharmaceutical composition comprises a FoxM1 inhibitor and paclitaxel. In yet certain other embodiments the pharmaceutical composition comprises a FoxM1 inhibitor and trastuzumab and paclitaxel. In particular embodiments the FoxM1 inhibitor comprises an inhibitory P19ARF peptide including without limitation a peptide having the sequence of SEQ ID NO:6 or SEQ ID NO:7. In other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In other embodiments, the FoxM1 inhibitor comprises a thiazole antibiotic, including without limitation siomycin A or thiostrepton. In yet other embodiments the FoxM1 inhibitor is an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP). These embodiments are suitable for use in every aspect of the invention described herein.

In another aspect, the invention provides compositions or kits for inhibiting tumor growth comprising a combination of a FoxM1 inhibitor and either trastuzumab or paclitaxel. In certain particular embodiments the compositions or kits comprise a FoxM1 inhibitor and trastuzumab. In certain other embodiments the compositions or kits comprise a FoxM1 inhibitor and paclitaxel. In yet other certain embodiments the compositions or kits comprises a FoxM1 inhibitor and trastuzumab and paclitaxel. In further embodiments, the FoxM1 inhibitor comprises an inhibitory P19ARF peptide including without limitation a peptide having the sequence of SEQ ID NO:6 or SEQ ID NO:7. In certain other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In further embodiments, the FoxM1 inhibitor comprises a thiazole antibiotic, specifically siomycin A or thiostrepton. In yet other embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In another aspect, the invention provides methods for treating breast cancer in a patient comprising the step of administering to a patient in need thereof a pharmaceutical composition comprising a combination of a FoxM1 inhibitor and either trastuzumab or paclitaxel or both, and a pharmaceutically acceptable excipient, diluent or carrier, wherein the breast cancer cell is HER2/ErbB2 positive. In certain particular embodiments the pharmaceutical composition comprises a FoxM1 inhibitor and trastuzumab. In certain other embodiments the pharmaceutical composition comprises a FoxM1 inhibitor and paclitaxel. In yet other embodiments the pharmaceutical composition comprises a FoxM1 inhibitor and trastuzumab and paclitaxel. In yet another aspect, the invention provides methods for treating breast cancer in a patient comprising the step of administering to a patient in need thereof a FoxM1 inhibitor and either trastuzumab or paclitaxel or both trastuzumab and paclitaxel, wherein the breast cancer cell is HER2/ErbB2 positive. In embodiments of the above aspects, the breast cancer is resistant to trastuzumab treatment and/or paclitaxel treatment. In other embodiments the breast cancer is sensitive to trastuzumab treatment and/or paclitaxel treatment. In certain other embodiments, the breast cancer is sensitive to trastuzumab treatment and resistant to paclitaxel treatment; and in yet other embodiments, the breast cancer is resistant to trastuzumab and sensitive to paclitaxel treatment. In certain particular embodiments, the FoxM1 inhibitor comprises an inhibitory P19ARF peptide including without limitation a peptide having the sequence of SEQ ID NO:6 or SEQ ID NO:7. Yet in other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In certain other embodiments, the FoxM1 inhibitor comprises a thiazole antibiotic, including without limitation siomycin A or thiostrepton. In yet other certain embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In a further aspect, the invention provides methods for treating HER2/ErbB2 positive cancer in a patient comprising the steps of (a) obtaining a breast cancer tissue sample from a patient in need of the treatment, wherein the breast cancer tissue sample is HER2/ErbB2 positive; (b) detecting FoxM1 expression in the breast cancer tissue sample using a reagent that specifically detects FoxM1; and (c) administering to the patient a FoxM1 inhibitor and either trastuzumab or paclitaxel or both trastuzumab and paclitaxel if FoxM1 expression is detected in the breast cancer tissue sample. In certain particular embodiments, the FoxM1 expression is detected in the nucleus of the cells of the breast cancer tissue sample. In other embodiments, the method further comprises the steps of obtaining a control breast tissue sample, detecting FoxM1 expression in the control breast tissue sample, wherein in step (c) a FoxM1 inhibitor is administered to the patient with trastuzumab or paclitaxel if FoxM1 expression is higher in the breast cancer tissue sample than in the control breast tissue sample. In yet other embodiments, step (c) includes administering to the patient a FoxM1 inhibitor and trastuzumab and paclitaxel. In certain embodiments, the FoxM1 inhibitor comprises an inhibitory P19ARF peptide including without limitation a peptide having the sequence of SEQ ID NO:6 or SEQ ID NO:7. In other certain embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In other certain embodiments, the FoxM1 inhibitor comprises a thiazole antibiotic, including without limitation siomycin A or thiostrepton. In yet other certain embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In yet another aspect, the invention provides methods of identifying trastuzumab-resistant and/or paclitaxel-resistant breast cancer in a patient, wherein the breast cancer is HER2/ErbB2 positive, comprising the steps of (a) obtaining a breast cancer tissue sample from a patient having breast cancer that is HER2/ErbB2 positive; and (b) detecting FoxM1 expression in the breast cancer tissue sample using a reagent that specifically detects FoxM1, wherein detection of FoxM1 expression in the breast cancer tissue sample indicates that the breast cancer is resistant to trastuzumab treatment. In particular embodiments, FoxM1 expression is detected in the nucleus of the cancer cell. In other embodiments, the method further comprises the steps of obtaining a control breast tissue sample, and detecting FoxM1 expression in the control breast tissue sample, wherein the breast cancer is resistant to trastuzumab treatment and/or paclitaxel treatment if FoxM1 expression in the breast cancer tissue sample is greater than FoxM1 expression in the control breast tissue sample. In certain embodiments, the reagent comprises one or more FoxM1 specific primers, and the level of FoxM1 expression is determined by reverse-transcriptase polymerase chain reaction (RT-PCR). In other certain embodiments the reagent is a FoxM1 specific antibody and the level of FoxM1 expression is determined by an immunoassay.

In yet another aspect, the invention provides methods of reducing the risk of developing trastuzumab resistance and/or paclitaxel resistance in a patient with breast cancer comprising the step of administering to a patient in need thereof a FoxM1 inhibitor, wherein the breast cancer is HER2/ErbB2 positive. In a further aspect the invention provides methods of treating paclitaxel-resistant breast tumor in a patient comprising the step of administering to a patient in need thereof a FoxM1 inhibitor and paclitaxel, wherein the combination of the FoxM1 inhibitor and paclitaxel effectively inhibits paclitaxel-resistant breast tumor. In yet another aspect the invention provides methods of treating trastuzumab-resistant breast tumor in a patient comprising the step of administering to a patient in need thereof a FoxM1 inhibitor and trastuzumab, wherein the combination of the FoxM1 inhibitor and trastuzumab effectively inhibits trastuzumab-resistant breast tumor, and wherein the breast tumor is HER2/ErbB2 positive. In certain embodiments, the FoxM1 inhibitor comprises an inhibitory P19ARF peptide including without limitation a peptide having the sequence of SEQ ID NO:6 or SEQ ID NO:7. In certain other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In other embodiments, the FoxM1 inhibitor comprises a thiazole antibiotic, including without limitation siomycin A or thiostrepton. In yet other embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In another aspect, the invention provides methods of treating cancer in a patient comprising administering to a patient in need thereof a FoxM1 inhibitor and paclitaxel. In yet another aspect, the invention provides methods of reducing the risk of developing paclitaxel-resistance in a cancer patient comprising the step of administering to a patient in need thereof a FoxM1 inhibitor. In certain embodiments, the patient is administered a FoxM1 inhibitor and paclitaxel.

In another aspect, the invention provides methods of treating paclitaxel-resistant cancer in a patient comprising the steps of (a) obtaining a cancer tissue sample from a patient in need of the treatment; (b) detecting FoxM1 expression in the cancer tissue sample using a reagent that specifically detects FoxM1; (c) obtaining a control tissue sample; and (d) detecting FoxM1 expression in the control tissue sample, wherein a FoxM1 inhibitor is administered to the patient with paclitaxel if FoxM1 expression in the cancer tissue sample is greater than FoxM1 expression in the control tissue sample. In certain embodiments the reagent comprises one or more FoxM1 specific primers, and the level of FoxM1 expression is determined by reverse-transcriptase polymerase chain reaction (RT-PCR). In certain other embodiments, the reagent is a FoxM1 specific antibody and the level of FoxM1 expression is determined by an immunoassay. In particular embodiments of the invention the cancer is ovarian cancer, breast cancer, small cell lung cancer, non-small cell lung cancer, colorectal cancer, malignant peripheral nerve sheath tumors, cervical cancer, leukemia, prostate, Kaposi's sarcoma, metastatic melanoma, pancreatic cancer, head and neck tumors, meningiomas, basal cell carcinoma, and gliomas. In certain particular embodiments, the cancer is ovarian cancer, breast cancer, small cell lung cancer, non-small cell lung cancer, or Kaposi's sarcoma. In certain particular embodiments, the FoxM1 inhibitor comprises an inhibitory P19ARF peptide including without limitation a peptide having the sequence of SEQ ID NO:6 or SEQ ID NO:7. In certain other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In other embodiments, the FoxM1 inhibitor comprises a thiazole antibiotic, including without limitation siomycin A or thiostrepton. In yet other embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In another aspect, the invention provides methods of identifying paclitaxel-resistant cancer in a patient comprising the steps of (a) obtaining a cancer tissue sample from a patient (b) detecting FoxM1 expression in the cancer tissue sample using a reagent that specifically detects FoxM1, wherein detecting FoxM1 expression in the cancer tissue sample indicates that the cancer is resistant to paclitaxel treatment. In particular embodiments the FoxM1 expression is detected in the nucleus of the cells in the cancer tissue sample. In other embodiments, the method further comprises the steps of obtaining a control tissue sample, and detecting FoxM1 expression in the control tissue sample, wherein the cancer is resistant to paclitaxel treatment if FoxM1 expression in the cancer tissue sample is greater than FoxM1 expression in the control tissue sample. In certain embodiments the reagent comprises one or more FoxM1 specific primers, and the level of FoxM1 expression is determined by reverse-transcriptase polymerase chain reaction (RT-PCR). In other certain embodiment, the reagent is a FoxM1 specific antibody and the level of FoxM1 expression is determined by an immunoassay. In certain embodiments, the FoxM1 inhibitor comprises an inhibitory P19ARF peptide including without limitation a peptide having the sequence of SEQ ID NO:6 or SEQ ID NO:7. In other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In certain other embodiments, the FoxM1 inhibitor comprises a thiazole antibiotic, including without limitation siomycin A or thiostrepton. In yet other embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In yet another aspect, the invention provides methods of promoting breast tumor cell differentiation by reducing the FoxM1 activity or level of FoxM1 expression comprising the step of contacting the breast tumor with a FoxM1 inhibitor. In another aspect, the invention provides methods of promoting breast tumor cell differentiation that reduces GATA3 promoter methylation comprising the step of contacting the breast tumor with a FoxM1 inhibitor. In a further aspect, the invention provides methods of promoting breast tumor cell differentiation that reduces interactions between FoxM1 and Rb interaction comprising the step of contacting the breast tumor cell with a FoxM1 inhibitor. In certain embodiments, the breast tumor cell proliferation is inhibited by increased differentiation. In other certain embodiments, the breast tumor cell is contacted with the FoxM1 inhibitor when a patient with a breast tumor is administered the FoxM1 inhibitor. In certain embodiments, the FoxM1 inhibitor comprises an inhibitory P19ARF peptide including without limitation a peptide having the sequence of SEQ ID NO:6 or SEQ ID NO:7. In other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In certain other embodiments, the FoxM1 inhibitor comprises a thiazole antibiotic, including without limitation siomycin A or thiostrepton. In yet other embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In yet another aspect, the invention provides uses of a combination of a FoxM1 inhibitor together with trastuzumab or paclitaxel, present in a therapeutically effective amount, for the preparation of a medicament for inhibiting breast tumor growth in a mammal. In certain particular embodiments the composition comprises a FoxM1 inhibitor and trastuzumab. In certain other embodiments the composition comprises a FoxM1 inhibitor and paclitaxel. In yet other embodiments the composition further comprises a FoxM1 inhibitor and trastuzumab and paclitaxel. In certain embodiments, the FoxM1 inhibitor comprises an inhibitory P19ARF peptide including without limitation a peptide having the sequence of SEQ ID NO:6 or SEQ ID NO:7. In other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In certain other embodiments, the FoxM1 inhibitor comprises a thiazole antibiotic, including without limitation siomycin A or thiostrepton. In yet other embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

In a further aspect, the invention provides compositions for use in the inhibition of breast tumor growth in a mammal, wherein the compositions comprise a FoxM1 inhibitor and further comprises trastuzumab or paclitaxel. In certain particular embodiments the composition comprises a FoxM1 inhibitor and trastuzumab. In certain other embodiments the composition comprises a FoxM1 inhibitor and paclitaxel. In yet other embodiments the composition comprises a FoxM1 inhibitor and trastuzumab and paclitaxel. In certain embodiments, the FoxM1 inhibitor comprises an inhibitory P19ARF peptide including without limitation a peptide having the sequence of SEQ ID NO:6 or SEQ ID NO:7. In other embodiments, the FoxM1 inhibitor comprises a FoxM1-specific siRNA including without limitation a polynucleotide having the sequence of SEQ ID NO:8, SEQ ID NO:9, SEQ ID NO:10, or SEQ ID NO:11. In certain other embodiments, the FoxM1 inhibitor comprises a thiazole antibiotic, including without limitation siomycin A or thiostrepton. In yet other embodiments the FoxM1 inhibitor comprises an antioxidant including without limitation N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

Specific embodiments of the present invention will become evident from the following more detailed description of certain preferred embodiments and the claims.

### BRIEF DESCRIPTION OF THE DRAWINGS

FIGS. 1A-1C demonstrate that overexpression of FoxM1 renders multiple HER2/ErbB2 amplified (or HER2/ErbB2 overexpressing) cell lines resistant to the effects of HERCEPTIN treatment. FIG. 1A shows the response of SKBR3, MDA-MB-453, and BT474 cell lines to HERCEPTIN tested by colony forming assay. Specifically, FIG. 1A shows bar graphs of the number of colonies of pBabe or pBabe-FoxM1-infected cells treated continuously with 10 ug/ml HERCEPTIN for 14 days, as a percentage of untreated cell lines and a photograph showing representative wells for SKBR3. FIG. 1B shows graphs of percentage changes in G1 phase in cell lines stably infected with either pBabe or FoxM1 following treatment with 10 ug/ml of HERCEPTIN for 48 hours. Inset shows a picture of relative protein expression in FoxM1 versus pBabe stable cell lines. FIG. 1C presents a graph showing the percentage of BrdU positive cells compared to DAPI positive cells in SKBR3-pBabe and FoxM1 lines either untreated or treated for 72 hours with HERCEPTIN. 500 cells in each experiment were counted. Average values are shown above error bars and representative microphotographs of cells are shown below the graph.

FIGS. 2A-2C demonstrate that SKBR3-FoxM1 cell lines fail to accumulate p27 after treatment with HERCEPTIN. FIG. 2A shows photographs of western blots of FoxM1 and p27 levels in SKBR3-pBabe and FoxM1 cell lines treated with increasing doses of HERCEPTIN for 48 hours. FIG. 2B shows photographs of western blots of FoxM1 and p27 levels for SKBR3 stable cell lines treated with 10 ug/ml of HERCEPTIN for 24, 48, and 72 hours. FIG. 2C shows photographs of western blots of FoxM1 and p27 levels in SKBR3-pBabe cells treated with 10 ug/ml of IgG for indicated periods of time.

FIGS. 3A-3C demonstrate that FoxM1 expression is higher in resistant lines and that targeted inhibition of FoxM1 can resensitize the cells to HERCEPTIN. FIG. 3A presents photographs of western blots showing FoxM1 protein levels in SKBR3, BT474, and MDA-MB-453 parental and resistant lines obtained by continuously culturing in 5 ug/ml of HERCEPTIN for six months. Quantification of FoxM1 bands by Image J is shown above the blots, using untreated parental lines for normalization. FIG. 3B presents representative images of DNA gel electrophoresis results showing target gene expression levels measured by semi-quantitative RT-PCR using cDNA from either parental or resistant SKBR3 cells. Quantification normalized to GAPDH is shown above each image. FIG. 3C shows the number of parental and resistant SKBR3 and MDA-MB-453 cells after HERCEPTIN treatment as a percentage of corresponding untreated cells, wherein all the cells were transfected with either control or FoxM1 specific siRNA.

FIGS. 4A-4D demonstrate that FoxM1 expression induces resistance to TAXOL by increasing stathmin expression and activity. FIG. 4A, The top panel is a bar graph showing numbers of viable cells determined by luminescent measurement of ATP in SKBR3-pBabe and FoxM1 lines treated with 0.1 uM of TAXOL for 7 days. The bottom panel is a line graph measuring cell viability by a luminescence assay where SKBR3 parental cells were treated with control siRNA or FoxM1-specific siRNA for 72 hours followed by TAXOL treatment at indicated doses for 24 hours. FIG. 4B shows photographs of western blots of α-tubulin in polymerized and soluble tubulin fractions isolated by centrifugation from untreated and treated SKBR3-pBabe and FoxM1 cell lines. Western blot analysis was used to assay α-tubulin and 13-tubulin ratios in the polymerized and soluble fractions. Relative percentages are shown above each blot. FIG. 4C shows stathmin RNA levels in SKBR3 pBabe and FoxM1 lines measured by RT-PCR. Values were normalized against cyclophilin. The inset shows stathmin protein expression in pBabe and FoxM1 cells by western blot analysis. FIG. 4D shows representative PCR results from a chromatin immunoprecipitation assay (ChIP) performed in SKBR3 cells using an antibody specific to FoxM1 or a non-specific IgG as a control. Also shown is a diagram of the region amplified during ChIP (SEQ ID NO:14).

FIGS. 5A-5C demonstrate that FoxM1 protects cells against treatment with HERCEPTIN and TAXOL in combination. FIG. 5A shows a graph indicating number of SKBR3 cells as a percentage of untreated cells where the cells were pretreated with 10 ug/ml of HERCEPTIN for 3 days followed by 0.1 uM of TAXOL for 7 days in the presence of HERCEPTIN. FIG. 5B shows the number of surviving SKBR3 parental cells, as a percentage of untreated cells, treated with control or FoxM1 siRNA for 72 hours followed by 10 ug/ml of HERCEPTIN for 3 days. Equal numbers of cells were treated for 24 hours with increasing amounts of TAXOL and cell viability was measured by an ATP luminescence assay. FIG. 5C shows graphs of quantification of MDA-MB-453 and BT474 cells that were either left untreated or pre-treated in 10 ug/ml HERCEPTIN for 72 hours followed by 0.1 μM TAXOL treatment for 4 hours. Each graph shows quantification of triplicates from three separate experiments. Also shown are photographs of representative wells of SKBR3-pBabe and FoxM1 cells with or without drug treatment.

FIGS. 6A-6C demonstrate that targeted inhibition of FoxM1 with an ARF-peptide overcame HERCEPTIN resistance and sensitized pBabe or FoxM1 cells to HERCEPTIN treatment. FIGS. 6A and 6B are graphs showing quantitative colony forming assay of parental or resistant SKBR3 and MDA-MB-453 cells treated with either ARF-peptide or mutant peptide (2 μM). FIG. 6C shows bar graphs of surviving SKBR3-pBabe and FoxM1 cells, as a percentage of untreated cells, treated with either mutant or ARF-peptide for three days. Also shown below the graphs are images of representative wells of cells from such colony-forming assays.

FIGS. 7A and 7B show FoxM1 expression in human breast tumors. FIG. 7A is a graph showing microarray data from Oncomine sorted by tumor grade and FoxM1 fold change from normal expression. FIG. 7B shows images of wildtype tissue stained with a FoxM1 sense or antisense probe by in situ hybridization and immunostained with smooth muscle actin (SMA) or cytokeratin 18. Scale bar represents 100 μM.

FIGS. 8A-8F show FoxM1 expression in tumor and normal tissue. FIG. 8A shows FoxM1 expression in 200 samples of invasive ductal carcinoma by using Oncomine analysis. Samples were organized by grade and fold-change of FoxM1 RNA from normal was graphed using a box plot *p<10⁻⁶. FIG. 8B shows representative images of immunohistochemistry analysis of FoxM1 in normal human mammary tissue as well as grade 1, grade 2, and grade 3 human breast carcinomas. Scale bar represents 200 μm. FIG. 8C is a graph showing levels of FoxM1 RNA determined by semi-quantitative RT-PCR and FIG. 8D is a photograph of western blot showing FoxM1 protein levels. For FIGS. 8C and 8D all samples were collected from inguinal mammary glands at various developmental stages: 5 weeks (puberty), 8 weeks (virgin adult), P6, P18 (early and late pregnancy), L10 (lactation), and 16 (involution). 4-7 mice were used for each stage. FIG. 8E are photomicrographs of mouse mammary glands from each stage and stained for FoxM1 expression using 3,3′-diaminobenzidine (DAB) and hemetoxylin counterstain. FIG. 8F shows bar graphs depicting expression of CK18, SMA, and FoxM by quantitative RT-PCR. Data is normalized to the stem cell population, *p<10⁻⁴ **p<0.05.

FIGS. 9A-9E show results demonstrating that FoxM1 deletion leads to an expansion of differentiated luminal cells. FIG. 9A shows results of FoxM1 expression in different type of cells using RT-PCR, *p<0.01 **p<10⁻³. FIG. 9B shows images of whole mount of inguinal mammary glands from transgenic mice stained with carmine alum stain 15 days after doxycycline treatment. Enlarged images of the boxed regions are shown at higher magnification (3×) to the right. FIG. 9C shows images of Hemetoxylin and Eosin staining as well as immunohistochemistry of FoxM1, cytokeratin 18, and estrogen receptor alpha after 15 days of treatment. Scale bar represents 100 μm. FIG. 9D shows flow cytometry analysis of stem cells, luminal progenitors, and differentiated luminal cells from transgenic mice. A representative plot is shown with cell percentages displayed in each quadrant. Percentage change from four animals is graphed below, *p<0.04 **p<0.05 ***p<0.03. FIG. 9E shows RNA levels of markers of luminal differentiation (estrogen receptor alpha, amphiregulin, cytokeratin 18, and cadherin 11) by quantitative RT-PCR normalized to 18S RNA.

FIGS. 10A-10E demonstrate that over-expression of FoxM1 in mammary gland results in an expansion of progenitors and a loss of differentiation markers. FIG. 10A is a schematic representation of experimental design. FIG. 10B shows images of green fluorescent protein (GFP) staining of whole mount of mouse mammary glands. Boxed areas are shown in the inset at higher magnification (3×). FIG. 10C shows microphotographs of Hemetoxylin and Eosin staining and immunohistochemistry using different antibodies in GFP and FoxM1-GFP glands. Specifically, representative sections from six mice stained for smooth muscle actin (SMA), cytokeratin 18, and estrogen receptor alpha immunostaining are shown. Scale bar represents 100 μm. FIG. 10D shows images of CD61 immunohistochemistry. Enlarged images of GFP and GFP-FoxM1 mice are displayed in the right panel. FIG. 10E shows analysis of mammary stem cells, luminal progenitor, and luminal cell pools performed in glands obtained from GFP or FoxM1-GFP expressing mice. Representative dot plots are shown with percentages listed in each box. The bottom panel provides quantification from four mice. The change in percentage of each population is shown relative to the GFP control in the same animal, *p<0.03 **p<0.04 ***p<0.003. FIG. 10F shows RNA levels of estrogen receptor alpha, cytokeratin 18, amphiregulin, and cadherin 11 in GFP and GFP-FoxM1 glands measured by quantitative RT-PCR analysis. *p<10⁻⁴ **p<0.001 ***p<0.05.

FIGS. 11A and 11B shows images of mammary gland sections from GFP or GFP-FoxM1 expressing mice. FIG. 11A shows images of mammary gland sections from GFP-FoxM1 expressing mice stained with hemetoxylin and eosin. FIG. 11B presents images of p63 staining of both GFP and GFP-FoxM1 mice, which show a normal negative staining pattern for p63 in both GFP and GFP-FoxM1 mice. Scale bar, 100 μM.

FIGS. 12A-12E show results demonstrating FoxM1 as a negative regulator of GATA-3 in vivo. FIG. 12A shows photographs of western blots of FoxM1 and GATA-3 protein levels in WAP-rtTA-Cre, FoxM1 FL/+ (control) and WAP-rtTA-Cre, FoxM1 FL/FL as well as GFP (control) and GFP-FoxM1 expressing animals. Alpha tubulin is shown as a loading control. FIG. 12B shows images of immunohistochemical staining of GATA-3 expression by DAB and hematoxylin counterstain. FIG. 12C shows results of RT-PCR for GATA-3 expression. Flow cytometry markers were used to sort stem cells, luminal progenitors, and differentiated cells. These populations were analyzed by RT-PCR for GATA-3 expression. The left panel shows data from FoxM1 deleted samples, *p<10⁻⁵. Relative GATA-3 expression as compared to control samples is displayed. The right panel shows data from animals over-expressing FoxM1 in the mammary gland. Four animals were used for each experiment, *p<10⁻³ **p<0.01 ***p<0.05. FIG. 12D presents graphs showing relative binding of FoxM1 antibody to sequences in the GATA3 promoter regions over an IgG control, *p<10⁻⁹ **p<10⁻⁴ ***p<0.01. Also shown is a diagram of the GATA-3 promoter. FIG. 12E shows graphs summarizing the flow cytometry data from control, GATA-3, FoxM1, and FoxM1-GATA-3 expressing mice. Each group contains three mice and the percentage of each cell type is graphed. For each group, p-values are calculated as compared to control animals. Photographs of western blots showing protein levels are shown to the right, *p<0.05 **p<0.01.

FIGS. 13A-13E show results demonstrating that FoxM1 transcriptional repression of GATA-3 is methylation-dependent. FIG. 13A shows the FoxM1 and GATA-3 expression in human breast cancers. The fold changes from normal are graphed and the heat map of individual samples is shown above the graphs, *p<10⁻³ **p<10⁻⁵ ***p<10⁻¹¹. FIG. 13B shows semi-quantitative PCR results for chromatin immunoprecipitation assay of FoxM1 binding to the GATA-3 promoter in human cell line MDA-MB-453. Also shown is a diagram of the GATA-3 promoter. FIG. 13C depicts RT-PCR results of GATA-3 expression normalized to GAPDH. (*p<0.01) MDA-MB-453 cells transfected with FoxM1 and 4 hours later, either vehicle (PBS) or 1 uM of the methyltransferase inhibitor 5′azacytidine was added to each plate. FIG. 13D shows images of western blots of immunoprecipitation results indicating the association of FoxM1 with DNMT3a and DNMT3b in cells transiently transfected with FoxM1 and myc tag alone or myc tagged DNMT3a or DNMT3b. FIG. 13E shows a bar graph depicting binding of DNMT3b to the FoxM1 binding sites in the GATA-3 promoter. The results have been normalized to the binding of a non-specific IgG and relative binding is shown, *p<0.01, **p<0.05.

FIGS. 14A-14C shows results demonstrating interaction between FoxM1 and Rb1 (i.e., Rb). FIG. 14A is an image of western blot demonstrating the binding of endogenous FoxM1 to Rb1 in MDA-MB-453 cells. FIG. 14B depicts results of western blot analysis of protein lysates from cells grown in media treated with doxycycline (+Dox) and without addition of doxycycline (−Dox). FIG. 14C shows microphotographs of phase contrast and florescent microscopy of cells grown in the presence or absence of doxycycline.

FIG. 15A-15E presents results demonstrating that methylation of GATA3 promoter by FoxM1 is Rb-dependent. FIG. 15A shows GATA-3 expression levels measured by RT-PCR normalized to GAPDH, *p<0.05 **p<0.001. FIG. 15B shows Rb binding to the GATA-3 promoter determined by real-time PCR, *p<0.05 **p<10⁻⁴. FIG. 15C shows methylation-specific PCR analysis of the GATA-3 promoter in the presence and absence of FoxM1 expression in Tet-off shRNA cell lines. FIG. 15D shows the results of flow cytometry of stem cells, luminal progenitors, and differentiated cells from mice expressing scrambled shRNA, Rb-targeting shRNA, FoxM1, or both FoxM1 and Rb-targeting shRNA. Panel to the right shows semi-quantitative RT-PCR of FoxM1, GATA-3 and Rb expression. Cyclophilin is shown as a loading control, *p<10⁻⁴ **p<0.01.

### DETAILED DESCRIPTION OF THE INVENTION

The invention provides methods for treating breast cancer, especially HER2/ErbB2 positive breast cancer, that are not hampered by the limitations existing for conventional treatment. In particular, these methods are able to treat breast cancer using a combination of a FoxM1 inhibitor and trastuzumab (HERCEPTIN) or a FoxM1 inhibitor and paclitaxel (TAXOL), wherein trastuzumab and paclitaxel can each optionally be effectively used at suboptimal amounts, i.e. amounts lower than the currently clinically recommended amounts (thereby, inter alia, reducing side effects associated with such treatment). Advantageously, the inventive methods can overcome, or reduce the risk of developing, breast cancer resistance to trastuzumab and/or paclitaxel, one of the significant drawbacks of trastuzumab and paclitaxel therapy for treating breast cancer.

All molecular biology and DNA recombination techniques described herein are well known to one of ordinary skill in the art and further described in reference books such as Molecular Cloning: A Laboratory Manual (Sambrook, et al., 1989, Cold Spring Harbor Laboratory Press), which is incorporated herein by reference for any purposes. All references cited throughout the application are herein incorporated by reference in their entireties for any and all purposes.

As used herein, the singular forms “a”, “an” and “the” include plural referents unless the context clearly dictates otherwise.

HERCEPTIN (trastuzumab) is a humanized monoclonal antibody directed to the extracellular domain of HER2/ErbB2. The binding of trastuzumab with HER2/ErbB2 blocks or reduces downstream signal transduction that leads to cell growth; however, side effects of heart and lung problems, fever, nausea, vomiting, fatigue, low white and red blood cells, muscle pain and serious infusion reactions have been reported in patients receiving trastuzumab therapy. In addition, inherent and acquired resistance to trastuzumab in patients reduces the effectiveness of this antibody for breast cancer treatment.

Because the mechanisms of action of HERCEPTIN are not yet fully understood, it has been difficult in the field to explain the reasons why some patients are naturally resistant to HERCEPTIN and others have quickly developed resistance during treatment. Several hypotheses have been presented including loss of PTEN (phosphatase and tensin homologue), activation of alternative IGF-R signal transduction pathway, expression of ligands of the EGFR family and receptor masking or epitope inaccessibility (e.g., Valabrega et al., 2007, supra). There has not been a successful solution to restore sensitivity of target breast cancers to HERCEPTIN.

However, it was unexpectedly discovered by the inventors of the instant application that decreasing FoxM1 activity inter alia using FoxM1 inhibitors restored sensitivity to trastuzumab in HER2/ErbB2 positive cell. As shown in the examples disclosed herein, FoxM1 overexpression was associated with trastuzumab resistance in HER2/ErbB2 positive breast tumor cells, and inhibition of FoxM1 in those cells resensitized the cells to trastuzumab. To the best of the knowledge of the inventors, the instant application established for the first time the connection between FoxM1 levels and resistance to trastuzumab in HER2/ErbB2 positive cells, and demonstrated for the first time restoration of sensitivity to trastuzumab in the resistant cells by decreasing the levels or activity of FoxM1.

Accordingly, the instant invention provides improved and advantageous methods for treating HER2/ErbB2 positive breast tumor in a patient comprising the step of administering to a patient in need thereof a pharmaceutical composition comprising a FoxM1 inhibitor and trastuzumab. In certain particular embodiments, the breast cancer is resistant to trastuzumab. In certain other embodiments, the breast cancer is sensitive to trastuzumab. In particular, inhibition of FoxM1 activity by a FoxM1 inhibitor can overcome, and prevent cells from developing, resistance to trastuzumab. Thus, in another advantageous aspect, the invention provides methods of reducing the risk of developing trastuzumab resistance in a patient with HER2/ErbB2 positive breast cancer comprising the step of administering to a patient in need thereof a FoxM1 inhibitor and trastuzumab. In a further aspect, the invention provides methods of treating trastuzumab resistant HER2/ErbB2 positive breast cancer comprising the step of administering to a patient in need thereof a FoxM1 inhibitor and trastuzumab.

As used herein, the term “HER2/ErbB2 positive breast tumor cells” or “HER2/ErbB2 positive breast tissue sample” refers to breast tumor cells that express HER2/ErbB2 at a level higher than the breast cells or breast tissue from a control sample. HER2/ErbB2 positive status indicates that HER2/ErbB2 is expressed at elevated levels by events such as chromosomal amplification or upregulation of expression at the mRNA or protein level. Chromosome amplification can be determined by FISH (fluorescent in situ hybridization), and overexpression in the absence of amplification can be determined by IHC (immunohistochemistry). This can be done for example by using a commercially available kit such as HercepTest™ (DAKO), in which a standardized staining protocol and controls for each level of expression are provided. Scoring of the staining is based on a scale of 0-3. A score of 0 (or HER2/ErbB2 negative) indicates that less than 10% of the cells stain “faintly positive.” A score of 1 indicates greater than 10% stain “faintly positive.” A score of 2 indicates greater than 10% of cells stain “moderately positive,” and a score of 3 indicates “strong staining” in greater than 10% of cells. Samples with a score of 2-3 are considered HER2/ErbB2 positive.

“Treating” or “treatment” as used herein covers the treatment of a disease or disorder described herein, in a patient and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In certain particular embodiments, administering to a HER2/ErbB2 positive breast cancer patient who is resistant to trastuzumab treatment a FoxM1 inhibitor can inhibit and/or slow the progression of trastuzumab-resistant breast cancer.

“Preventing” or “reducing the risk of developing” a disease or condition as used herein refers to (i) inhibiting the onset of a disease or a condition in a patient who may be at risk of or predisposed to developing the disease or condition; and/or (ii) slowing the onset of the pathology or symptom of a disease or condition in a patient who may be at risk of or predisposed to developing the disease or condition. For example, administering to a HER2/ErbB2 positive breast cancer patient a FoxM1 inhibitor during the trastuzumab treatment regimen can reduce the risk of the patient in developing resistance to trastuzumab associated with trastuzumab therapy.

A “patient” or “subject” as used herein refers to a mammal, preferably a human, in need of the treatment of the claimed invention.

Trastuzumab is frequently administered to a patient in conjunction with other therapeutics such as the microtubule-stabilizing agent paclitaxel. It has been reported that HER2/ErbB2 positive cells can exhibit reduced sensitivity to paclitaxel (Azambuja et al., 2008, “HER-2 overexpression/amplification and its interaction with taxane-based therapy in breast cancer” Ann Oncol 19: 223-32; Yu et al., 1998, “Overexpression of ErbB2 blocks Taxol-induced apoptosis by upregulation of p21Cip1, which inhibits p34Cdc2 kinase” Mol Cell 2: 581-91). Further, paclitaxel resistance has been documented in every tumor type where paclitaxel is a cornerstone of treatment, including without limitation ovarian cancer, Kaposi's sarcoma, and non-small cell lung carcinoma. It was further surprisingly discovered by the inventors that elevated FoxM1 levels not only led to cell resistance to trastuzumab, but also protected the cells from paclitaxel-induced apoptosis and led to resistance to paclitaxel.

Thus, in certain particular embodiments, the invention provides methods of treating HER2/ErbB2 positive breast cancer in a patient comprising the step of administering to a patient in need thereof a FoxM1 inhibitor and paclitaxel. In certain embodiments, the breast cancer is resistant to paclitaxel. In other embodiments, the breast cancer is resistant to trastuzumab and paclitaxel. In certain other embodiments, the breast cancer is sensitive to paclitaxel, and the FoxM1 inhibitor reduces the level or activity of FoxM1, thereby reducing the risk of developing resistance to paclitaxel.

The invention in another aspect provides methods of treating cancer in a patient comprising administering to a patient in need thereof a FoxM1 inhibitor and paclitaxel. FoxM1 has been implicated in the growth, proliferation, or survival associated with, for example, malignant peripheral nerve sheath tumors (Yu et al., 2011, “Array-Based Comparative Genomic Hybridization Identifies CDK4 and FOXM1 Alterations as Independent Predictors of Survival in Malignant Peripheral Nerve Sheath Tumor” Clin Cancer Res 17:1924-1934), cervical cancer (Guan et al., 2011, “Expression and significance of FOXM1 in human cervical cancer: A tissue micro-array study,” Clin Invest Med 34:E1-E7), leukemia (Nakamura et al., 2010, “The FOXM1 transcriptional factor promotes the proliferation of leukemia cells through modulation of cell cycle progression in acute myeloid leukemia” Carcinogenesis 31:2012-21), prostate (Wang et al., 2011, “Down-regulation of Notch-1 is associated with Akt and FoxM1 in inducing cell growth inhibition and apoptosis in prostate cancer cells” J Cell Biochem 112:78-88), metastatic melanoma (Huynh et al., 2011, “FOXM1 expression mediates growth suppression during terminal differentiation of HO-1 human metastatic melanoma cells” J Cell Physiol 226:194-204), pancreatic cancer (Wang et al., 2010, “FoxM1 is a novel target of a natural agent in pancreatic cancer” Pharm Res 27:1159-68), head and neck tumors (Waseem et al., 2010, “Downstream targets of FOXM1: CEP55 and HELLS are cancer progression markers of head and neck squamous cell carcinoma” Oral Oncol 46:536-42), meningiomas (Laurendeau et al., 2010, “Gene expression profiling of the hedgehog signaling pathway in human meningiomas” Mol Med 16:262-70), basal cell carcinoma (Teh et al., 2002, “FOXM1 is a downstream target of Glil in basal cell carcinomas” Cancer Res 62:4773-80), and gliomas (Liu et al., 2006, “FoxM1B is overexpressed in human glioblastomas and critically regulates the tumorigenicity of glioma cells” Cancer Res 66:3593-602).

In a further aspect, the invention provides methods of reducing the risk of developing paclitaxel-resistance in a cancer patient comprising the step of administering to a patient in need thereof a FoxM1 inhibitor. Cancer types that can be treated by the inventive methods include without limitation ovarian cancer, breast cancer, small cell lung cancer, non-small cell lung cancer, colorectal cancer, malignant peripheral nerve sheath tumors, cervical cancer, leukemia, prostate, Kaposi's sarcoma, metastatic melanoma, pancreatic cancer, head and neck tumors, meningiomas, basal cell carcinoma, and gliomas. In certain particular embodiments, the cancer is ovarian cancer, breast cancer, small cell lung cancer, non-small cell lung cancer, or Kaposi's sarcoma.

It was also unexpectedly discovered by the instant inventors that, in the presence of a FoxM1 inhibitor, paclitaxel or trastuzumab effectively inhibited tumor growth at lower doses or achieved greater tumor inhibition effects at the same doses as compared to results obtained in the absence of a FoxM1 inhibitor. Advantageously, the claimed invention makes it possible to administer to a patient in need thereof trastuzumab and/or paclitaxel at suboptimal doses, i.e. doses that are less than the therapeutically effective amounts required when the drugs are administered, either alone or in combination, in the absence of a FoxM1 inhibitor. In accordance with the invention, in certain particular embodiments of all the aspects disclosed herein, trastuzumab is administered to a patient at a suboptimal amount or dose in conjunction with a FoxM1 inhibitor. In certain other particular embodiments, paclitaxel is administered at a suboptimal amount or dose in conjunction with a FoxM1 inhibitor. In certain other particular embodiments, both trastuzumab and paclitaxel are administered at suboptimal amounts or doses in conjunction with a FoxM1 inhibitor. The determination of a suitable suboptimal yet effective amount of HERCEPTIN or paclitaxel when administered in conjunction with a FoxM1 inhibitor is within the knowledge of a skill artisan or physician. In certain particular embodiments, the suboptimal amount of HERCEPTIN is initially less than 4 mg/kg/wk, followed by an amount of less than 2 mg/kg/wk. In certain other embodiments, the suboptimal amount is from 0.5 mg/kg/wk to 3 mg/kg, 1 mg/kg/wk to 2.5 mg/kg/wk, or 1.5 mg/kg/wk to 3 mg/kg/wk. In certain other particular embodiments, the suboptimal amount of paclitaxel is less than 175 mg/m², less than 135 mg/m², from 30-150 mg/m², from 50-130 mg/m², or from 70-100 mg/m².

Thus, as used herein the term “effective amount” or a “therapeutically effective amount” refers to an amount sufficient to achieve the stated desired result, for example, treating breast cancer or reducing the risk of developing trastuzumab resistance or paclitaxel resistance in a patient with breast cancer. A pharmaceutical composition in a therapeutically effective amount comprising a FoxM1 inhibitor, further comprising trastuzumab or paclitaxel means that the pharmaceutical composition when used as a whole provides a therapeutically effective amount for the desired outcome, whereas each individual active pharmaceutical ingredient can be present in suboptimal amounts. Thus, the invention provides methods of treating cancer, in particular trastuzumab-resistant and/or paclitaxel-resistant cancer, comprising administering to a patient in need thereof a combination of a FoxM1 inhibitor and either trastuzumab or paclitaxel or both trastuzumab and paclitaxel, wherein the combination effectively inhibits tumor growth.

In addition, the skilled worker will recognize that these embodiments of the invention are not limited to amounts that are formulated together in a single dose, but comprise any embodiments where the combination of dosages or amounts of FoxM1 and trastuzumab or paclitaxel or both are administered to a patient in need thereof in separate dosage forms and at times appropriate to have the desired therapeutic effect. For example, in certain embodiments, the FoxM1 inhibitor and trastuzumab and/or paclitaxel are administered to a patient at the same time. In certain other embodiments, the FoxM1 inhibitor and trastuzumab and/or paclitaxel are administered to a patient at different time. In additional embodiments, the FoxM1 inhibitor and trastuzumab and/or paclitaxel are provided in a single dose or dosage form. In yet other embodiments, the FoxM1 inhibitor and trastuzumab and/or paclitaxel are provided in separate doses or dosage forms.

The term “FoxM1 inhibitor” as used herein refers to a chemical compound or biological molecule that reduces expression of FoxM1 or inhibits FoxM1 activity in a cell. In certain embodiments of all aspects of the invention, the FoxM1 inhibitor comprises an inhibitory p19ARF peptide. Non-limiting exemplary inhibitory p19ARF peptides are disclosed in co-owned U.S. Pat. Nos. 7,635,673 and 7,799,896, which are incorporated herein by reference in their entireties.

The terms “peptide” and “polypeptide” both refer to a protein or a polymer of amino acids linked by peptide bonds. A peptide is generally shorter than a polypeptide; however, both peptide and polypeptide can be used to refer to a full-length protein or a fragment of the full-length protein.

In certain embodiments, the inhibitory p19ARF peptide comprises full-length p19ARF protein as shown in SEQ ID NO:1, also described in U.S. Pat. No. 6,407,062, which is herein incorporated by reference in its entirety. In certain particular embodiments, the inhibitory p19ARF peptide comprises a fragment of p19ARF protein, wherein the fragment comprises amino acid residues 26-44 of the p19ARF protein (SEQ ID NO:2). In certain embodiments, the inhibitory p19ARF peptide comprising a fragment of full-length p19ARF protein, wherein the fragment comprises amino acid residues of 26-44 of the full-length protein, and is about 19-80, about 20-60, or about 25-50 amino acids in length. Suitable inhibitory p19ARF peptide includes without limitation peptides having amino acid residues 26-44 (SEQ ID NO:2) and 26-55 (SEQ ID NO:3). In certain embodiments, the full-length p19ARF is used.

In certain particular embodiments, the p19ARF inhibitory peptide further comprises a cell-penetrating peptide covalently linked to the p19ARF peptide, either at the N- or C-terminus, but particularly at the N-terminus, to facilitate cellular uptake of the inhibitory peptide. In certain particular embodiments, the cell-penetrating peptide is covalently linked to the p19ARF peptide at the N-terminus Peptides that facilitate cellular uptake are well known in the art including without limitation the D-Arginine nona-peptide (SEQ ID NO:4) and the HIV TAT peptide (SEQ ID NO:5). Other suitable cell-penetrating peptides are known in the art and are contemplated for use in the instant invention. (See for example Okuyama et al., 2007, “Small-molecule mimics of an α-helix for efficient transport of proteins into cells” Nature Methods 4:153-159.) In certain embodiments, inhibitory p19ARF peptide has the sequence of SEQ ID NO:6. In certain particular embodiments, the p19ARF inhibitory peptide has the sequence of SEQ ID NO:7. In certain other embodiments, the full-length p19ARF covalently linked to a cell-penetrating peptide at the N-terminus is used.

In certain other embodiments, the FoxM1 inhibitor comprises an siRNA specific for FoxM1. Suitable FoxM1-specific siRNAs include, without limitation, polynucleotide having sequence of 5′-CAA CAG GAG UCU AAU CAA GUU-3′ (SEQ ID NO:8), 5′-GGA CCA CUU UCC CUA CUU UUU-3′ (SEQ ID NO:9), 5′-GUA GUG GGC CCA ACA AAU UUU-3′ (SEQ ID NO:10), or 5′-GCU GGG AUC AAG AUU AUU AUU-3′ (SEQ ID NO:11). In certain particular embodiments, the FoxM1-specific siRNA comprises a polynucleotide having sequence as set forth in SEQ ID NO:9. See U.S. Patent Application, Publication No. 2010-0098663, which is incorporated herein by reference in its entirety. It is understood by an ordinarily skilled artisan that the first 19 nucleotides of any one of SEQ ID NOs:8-11 are FoxM1-specific sequences, and the 3′ end UU overhang is not. In certain embodiments, suitable FoxM1 siRNAs may comprise the 19 FoxM1-specific nucleotides of any one of SEQ ID NOs:8-11, and additional FoxM1 sequence, with the UU at the 3′ end.

In yet other particular embodiments, the FoxM1 inhibitors suitable for use in the instant invention comprise a thiazole antibiotic, including but not limited to Siomycin A, thiostrepton, sporangiomycin, nosiheptide, multhiomycin, micrococcin or thiocillin. In certain particular embodiments, the thiazole antibiotic is siomycin A or thiostrepton. In certain further embodiments, the FoxM1 inhibitor is the EGFR inhibitor Gefitinib that targets FoxM1 (McGovern et al., 2009, “Gefitinib (Iressa) represses FOXM1 expression via FOXO3a in breast cancer” Mol Cancer Ther 8:582-91). In certain other embodiments, the FoxM1 inhibitor comprises an antioxidant such as N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP) (Part et al., 2009, “FoxM1, a critical regulator of oxidative stress during oncogenesis” EMBO 28:2908-2918). In certain other embodiments, the FoxM1 inhibitor comprises a proteasome inhibitor such as MG132 (Z-L-leucyl-L-leucyl-L-leucinal), MG115 (Z-L-leucyl-L-leucyl-L-norvalinal), VELCADE® (bortezomib, pyrazylcarbony-phenylalanyl-leucyl-boronate, Millennium Pharmaceuticals, Cambridge, Mass.), lactacystin, or PSI (N-benzyloxycarbony-Ile-Glu-(O-t-butyl)-Ala-leucinal) (SEQ ID NO:13), NPI-0052 (Salinsporamide-A), and ALLN (Acetyl-L-Leucyl-L-Leucyl-L-Norleucinal) (Bhat et al., 2009, “FoxM1 is a general target for proteasome inhibitors” PLoS One 4: e6593). In certain particular embodiments, the proteasome inhibitor is VELCADE®. See co-owned International patent application, Publication No. WO/2009/152462 and U.S. Patent Application Publication No. 2008-0152618, both of which are incorporated herein by reference in their entireties.

Nonlimiting examples of FoxM1 inhibitors described herein are suitable for use in all aspects and embodiments of the invention. It is within the knowledge of one skilled artisan or physician to choose a FoxM1 inhibitor and determine adequate amounts of the FoxM1 inhibitor for use in the instant invention.

In a further aspect, the invention provides methods of treating HER2/ErbB2 positive breast cancer in a patient comprising the steps of (a) obtaining a breast cancer tissue sample from a patient in need of the treatment, wherein the breast cancer tissue sample is HER2/ErbB2 positive; (b) detecting FoxM1 expression in the breast cancer tissue sample using a reagent that specifically detects FoxM1; and (c) administering to the patient a FoxM1 inhibitor and trastuzumab or paclitaxel if FoxM1 expression is detected in the breast cancer tissue sample. In another aspect, the invention provides methods of identifying trastuzumab-resistant or paclitaxel-resistant breast cancer in a patient, wherein the breast cancer is HER2/ErbB2 positive, comprising the steps of (a) obtaining a breast cancer tissue sample from a patient having breast cancer that is HER2/ErbB2 positive; and (b) detecting FoxM1 expression in the breast cancer tissue sample using a reagent that specifically detects FoxM1, wherein detection of FoxM1 expression in the breast cancer tissue sample indicates that the breast cancer is resistant to trastuzumab treatment. The level of FoxM1 expression in normal breast cell is very low or often undetectable. Thus, detection of FoxM1 in breast tumor cells, in particular detection of FoxM1 in the nucleus of the breast tumor cells, can serve as an indicator of aggressive tumor that are refractory to trastuzumab or paclitaxel treatment, alone or in combination.

FoxM1 expression can be detected by any suitable methods known in the art, including without limitation Northern blot analysis, RT-PCR, in situ hybridization and immunoassays. Nonlimiting examples of immunoassays include western blot analysis, immunofluorescent staining, and immunohistochemical staining. FoxM1-specific antibodies have been previously described (Major et al., 2004, “Forkhead box M1B transcriptional activity requires binding of Cdk-cyclin complexes for phosphorylation-dependent recruitment of p300/CBP coactivators” Mol Cell Biol 24: 2649-61) and are commercially available from sources such as Santa Cruz Biotechnology, Inc.

In certain particular embodiments, the methods disclosed herein further comprise the steps of obtaining a control breast tissue sample; and detecting FoxM1 expression in the control breast tissue sample, wherein the breast cancer is resistant to trastuzumab treatment or paclitaxel treatment if FoxM1 expression in the breast cancer tissue sample is greater than FoxM1 expression in the control breast tissue sample.

FoxM1 overexpression is detected not only in breast cancer, but also in a variety of cancer types, and paclitaxel resistance has been seen in different tumor types. In another aspect, the invention provides methods of treating paclitaxel-resistant cancer in a patient comprising the steps of (a) obtaining a cancer tissue sample from a patient in need of the treatment; (b) detecting FoxM1 expression in the cancer tissue sample using a reagent that specifically detects FoxM1; (c) obtaining a control tissue sample; (d) detecting FoxM1 expression in the control tissue sample; and (e) administering a FoxM1 inhibitor to the patient when FoxM1 expression in the cancer tissue sample is greater than FoxM1 expression in the control tissue sample. In yet another aspect, the invention provides methods of identifying paclitaxel-resistant cancer in a patient comprising the steps of (a) obtaining a cancer tissue sample from a patient; and (b) detecting FoxM1 expression in the cancer tissue sample using a reagent that specifically detects FoxM1, wherein detecting FoxM1 expression in the cancer tissue sample indicates that the cancer is resistant to paclitaxel treatment. In certain particular embodiments, FoxM1 expression is detected in the nucleus of the cells of the cancer tissue sample.

A “control breast tissue sample” as the term is used herein can be a normal, non-cancerous breast tissue sample obtained from a proximal or distal site of the breast tissue from a breast cancer patient. It can also be obtained from an individual that does not have breast cancer. Similarly, the term “control tissue sample” refers to a corresponding tissue sample from an individual that does not have cancer or a non-cancerous tissue sample from a proximal or distal site of the tissue from a cancer patient.

The mammary gland undergoes continuous cycles of proliferation, differentiation and apoptosis. The cellular plasticity is attributed to a stem cell population in the mammary gland (Kordon et al., 1998, “An entire functional mammary gland may comprise the progeny from a single cell” Development 125:1921-30). A pool of pluripotent stem cells in the mammary gland gives rise to lineage restricted progenitor cells that can be further differentiated into mature luminal or myoepithelial cells (Visvader, 2009, “Keeping abreast of the mammary epithelial hierarchy and breast tumorigenesis” Genes Dev 23:2563-77).

The zinc finger transcription factor GATA-3 is required for proper mammary gland development as well as maintenance of mature luminal cells (Kouros-Mehr et al., 2006, “GATA-3 links tumor differentiation and dissemination in a luminal breast cancer model” Cancer Cell 13:141-52; Asselin-Labat et al., 2007, “Gata-3 is an essential regulator of mammary-gland morphogenesis and luminal-cell differentiation” Nat Cell Biol 9:201-9). It has been shown that as tumor grade increases, GATA-3 expression is silenced by several mechanisms including DNA methylation (Yan et al., 2000, “CpG island arrays: an application toward deciphering epigenetic signatures of breast cancer” Clin Cancer Res 6:1432-8). FoxM1 expression has been shown to promote cell proliferation; however, FoxM1's direct role on regulating mammary gland differentiation has not been recognized in the art.

It was unexpectedly discovered by the inventors of the instant application that FoxM1 directly binds to the GATA3 promoter, promotes GATA3 promoter methylation in an Rb-dependent manner, and inhibits differentiation of the mammary progenitor cells. Further, as shown in the examples described herein, loss of FoxM1 in the adult gland leads to an increase in differentiated cells and a loss of progenitor pool cells. Accordingly, in a further aspect, the invention provides methods of promoting breast tumor cell differentiation by reducing the level of FoxM1 expression comprising the step of contacting the breast tumor with a FoxM1 inhibitor. In yet another aspect, the invention provides methods of promoting breast tumor cell differentiation that reduces GATA3 promoter methylation comprising the step of contacting the breast tumor with a FoxM1 inhibitor. In an additional aspect, the invention provides methods of promoting breast tumor cell differentiation that reduces interactions between FoxM1 and Rb interaction comprising the step of contacting the breast tumor cell with a FoxM1 inhibitor. This aspect of the invention provides unique methods for preventing or treating breast cancer cell growth with reduced cytotoxicity effects.

The pharmaceutical compositions of the invention may contain formulation materials for modifying, maintaining, or preserving, in a manner that does not hinder the physiological function of the active pharmaceutical ingredients, for example, pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobial compounds, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, betacyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; trimethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See REMINGTON'S PHARMACEUTICAL SCIENCES (18th Ed., A. R. Gennaro, ed., Mack Publishing Company 1990).

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention.

Administration routes for the pharmaceutical compositions of the invention include orally, through injection by intravenous, intraperitoneal, intramuscular, intravascular, intraarterial, intraportal, or intralesional routes; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

Examples that follow are illustrative of specific embodiments of the invention, and various uses thereof. They are set forth for explanatory purposes only, and are not to be taken as limiting the invention.

### EXAMPLES

### Cell Culture and Chemotherapeutic Agents

SKBR3 (breast adenocarcinoma), MDA-MB-453 (metastatic breast carcinoma), and BT474 (breast ductal carcinoma) cell lines were obtained from American Type Culture Collection (ATCC), Manassas, Va. Cells were cultured in RPMI 1640 (GIBCO) with 10% fetal bovine serum (FBS) and 100 U (units) penicillin and 100 ug streptomycin. Stable cell lines were generated by transfection of pBabe or pBabe-FoxM1 retroviral constructs followed by selection in puromycin (pBabe is obtainable from Addgene, Cambridge, Mass.). Control siRNA as well as siRNA specific to FoxM1 or Stathmin (Dharmacon, Lafayette. CO) were transfected using Lipofectamine (Invitrogen, Carlsbad, Calif.). Mutant and ARF peptide have been described previously (Gusarova et al., 2007, “A cell-penetrating ARF peptide inhibitor of FoxM1 in mouse hepatocellular carcinoma treatment” J Clin Invest 117: 99-111). See also co-owned U.S. Pat. Nos. 7,635,673 and 7,799,896, which are incorporated herein by reference in their entireties. Paclitaxel (Sigma) was dissolved in DMSO. HERCEPTIN (trastuzumab) was dissolved in sterile water (a gift from Genentech, San Francisco, Calif.).

A recombinant expression construct for expressing FoxM1, termed herein FoxM1-pcDNA3.1 was generated by PCR amplification and cloned into pcDNA3.1 (commercially available from Invitrogen), and the cloned sequence confirmed by sequencing. Myc tagged DNMT3a and 3b were a kind gift of Frederic Chedin. Retroviral scrambled shRNA and Rb shRNA constructs were purchased from Origene (Rockville, Md.). Plasmid transfection was done using FUGENE®6 (Roche, Indianapolis, Ind.). Control siRNA as well as siRNA specific to FoxM1 (Dharmacon) was transfected using Lipofectamine (Invitrogen).

### Example 1

### Effects of FoxM1 Overexpression on Trastuzumab Resistance

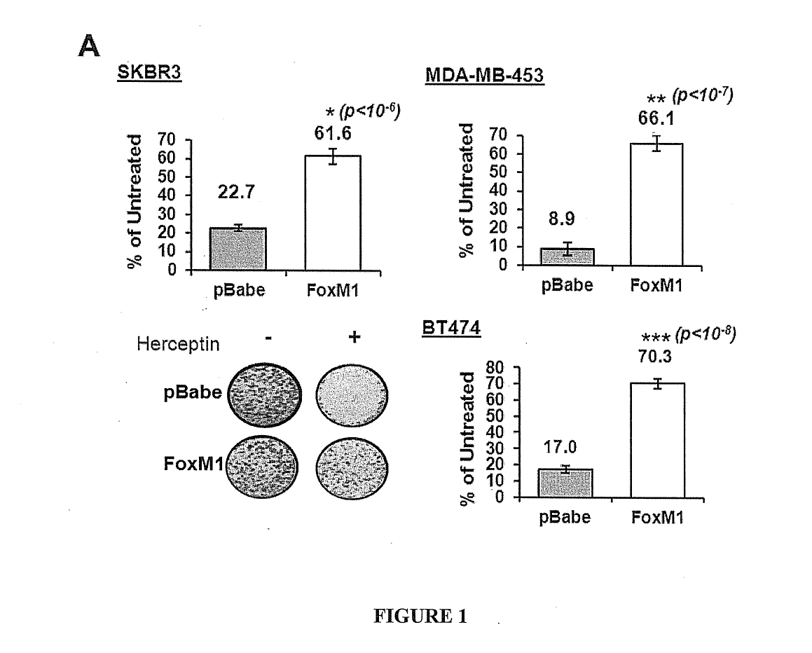

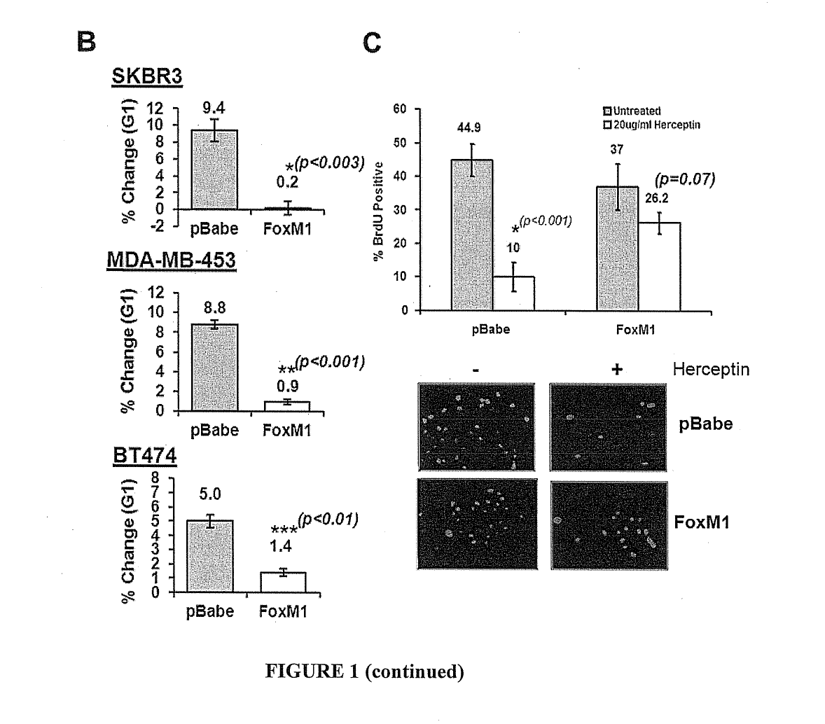

To investigate the effects of FoxM1 overexpression on trastuzumab resistance in breast tumor cells, FoxM1 expression cDNA construct was stably introduced into SKBR3, BT474, and MDA-MB-453 cell lines. All three cell lines have chromosomal amplification of HER2/ErbB2 and only the BT474 cell line expresses estrogen receptor. Drug sensitivity of the FoxM1 stably transfected cell lines was tested by colony formation assay. For colony forming assays, 3-5×10³ cells were plated in triplicate in 24-well plates. 24 hours later, cells were treated with trastuzumab (10 ug/ml) continuously for 14-17 days. After 14-17 days cells were fixed and stained with crystal violet. Quantification was done using Adobe Photoshop (Lehr et al., 1997, “Application of photoshop-based image analysis to quantification of hormone receptor expression in breast cancer,” J. Histochem Cytochem 45: 1559-65). All p-values were calculated using Student's t-test. FoxM1 overexpression resulted in a three- to seven-fold increase in colony number as compared to cells transfected with pBabe alone (FIG. 1A). The results provide evidence that FoxM1 confers cells resistance to trastuzumab.

The percentage of G1/S arrest in the cell cycle induced by trastuzumab (referred to as HERCEPTIN in the drawings contained herein) was measured by propidium iodide staining followed by flow cytometry (FACS) analysis. Cells were treated with trastuzumab (10 ug/ml) for 72 hours and cell cycle profiles examined. For cell cycle analysis, cells were trypsinized, pelleted, and resuspended in propidium iodide (PI) solution (50 ug/ml PI, 0.1 mg/ml RNaseA, 0.05% Triton-X). After 40 minutes of incubation at 37° C., cells were analyzed using a flow cytometer. Synchronization of MDA-MB-453 cells for cell cycle analysis was done by subjecting the cells to serum starvation (0.2% FBS) for 24 hours, followed by incubating the cells in medium containing 10% FBS for 6 hours, and addition of 5 ug/ml of aphidicolin (Calbiochem) for 16 hours.

The control pBabe lines showed a statistically significant increase in the number of cells in G1 after HERCEPTIN treatment, but the FoxM1-expressing cells did not exhibit any significant increase in the G1 population (FIG. 1B). None of the cell lines showed an increase in the sub-G1 population (data not shown), consistent with the understanding in the art that HERCEPTIN alone does not induce apoptosis (Nahta et al., 2004, “P27(kip1) down-regulation is associated with trastuzumab resistance in breast cancer cells,” Cancer Res. 64: 3981-6).

Further, incorporation of BrdU was measured in cells treated with HERCEPTIN (FIG. 1C). 5-Bromo-2-Deoxyuridine (BrDU, obtained from Sigma Chemical Co., St. Louis, Mo.; 10 μM) was added to the culture media. Cells were fixed and stained with mouse anti-BrdU antibody (1:250, Dako, Carpinteria, Calif.) followed by FITC-conjugated anti-mouse antibody (Dako) and DAPI (Molecular Probes/Invitrogen). Cell viability was measured using CellTiter-Glo Luminescent assay (Promega, Sunnyvale, Calif.), which measures the amount of oxygenated oxyluciferin directly correlated to the amount of ATP present. Upon treatment, SKBR3-pBabe showed a substantial (35%) reduction in the number of BrdU-positive cells. FoxM1-expressing cells did not show any significant decrease in BrdU-incorporation (FIG. 1C).

Taken together, these results indicate that FoxM1 expression was able to overcome the G1/S arrest and proliferation defect caused by HERCEPTIN, allowing cells to continue to grow in the presence of the drug.

### Example 2

### FoxM1 Prevents HERCEPTIN-Induced Accumulation of p27

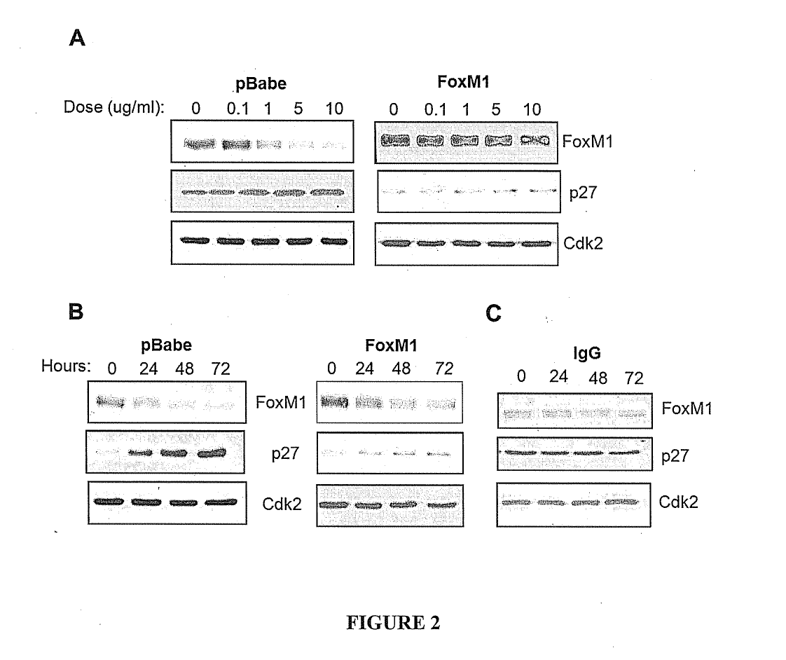

To investigate whether HERCEPTIN resistance observed in FoxM1 overexpressing cells resulted from a failure to accumulate p27, SKBR3-pBabe or FoxM1 expressing SKBR3 cells were treated with 10 ug/ml of HERCEPTIN for 0, 24, 48, or 72 hours or with increasing doses of HERCEPTIN (0, 0.1, 1, 5, and 10 μg/ml). Cell extracts were prepared in lysis buffer containing 1 mM EDTA, 0.15M NaCl, 0.05M Tris-HCl pH 7.5, and 0.5% Triton-X. Phosphatate Inhibitor Cocktail Set II (200 mM imidazole, 100 mM sodium fluoride, 115 mM sodium molybdate, 100 mM sodium orthovanadat, and 400 mM sodium tartrate, dehydrate, catalog No. 524625, Calbiochem) and protease inhibitor (Roche, catalog No. 11873580001, previously No. 115773860001) were added before each experiment. FoxM1 protein levels were determined by western blot analysis using a rabbit polyclonal antibody against FoxM1 previously described (Major et al., 2004, “Forkhead Box M1B transcriptional activity requires binding of Cdk-cycline complexes for phosphorylation-dependent recruitment of p300/CBP coactivators,” Mol Cell 24: 2649-61). Anti kip1/p27 (1:10,000, BD Biosciences), and anti-Cdk2 (1:200, Santa Cruz Biotech.) antibodies were also used. Quantification was performed using Image J software (NIH). The results as set forth in FIGS. 2A-2C show that in control SKBR3 cells, FoxM1 protein levels decreased and p27 levels accumulated after HERCEPTIN treatment. Interestingly, in SKBR3-FoxM1 cell lines, basal expression of p27 was lower than in SKBR3-pBabe cells and these levels remained low even after high-doses of HERCEPTIN (FIGS. 2A and 2B). Treatment with IgG did not cause changes in FoxM1 or p27 levels, therefore these effects were specific to HER2/ErbB2-related responses and not a general, non-specific antibody-induced response (FIG. 2C). Without being limited to particular mechanisms, these results show that FoxM1 conferred resistance to HERCEPTIN by preventing the accumulation of p27, the accumulation of which is required for HERCEPTIN induced G1/S arrest.

### Example 3

### Sensitizing Resistant Cells to HERCEPTIN Treatment

To determine whether cells resistant to HERCEPTIN could be resensitized to HERCEPTIN treatment, a cell line resistant to HERCEPTIN was generated. Parental SKBR3, MDA-MB-453, and BT474 lines were cultured continuously in 5 ug/ml of HERCEPTIN for six months. At the end of six months, the resistant cells grew at the same rate in the presence or absence of HERCEPTIN and the morphology of the cells was indistinguishable from the parent cells. The source of resistance in these lines was not uniform, as an increase in phosphorylated Akt was only observed in SKBR3 cells. FoxM1 levels in parental and resistant lines were assayed by western blot analysis. Extracts were prepared in lysis buffer containing 1 mM EDTA, 0.15M NaCl, 0.05M Tris-HCl pH 7.5, and 0.5% Triton-X. Phosphatate Inhibitor Cocktail Set II (Calbiochem) and protease inhibitor (Roche) were added before each experiment using the rabbit polyclonal antibody referenced above. Quantification was performed using Image J software (NIH).

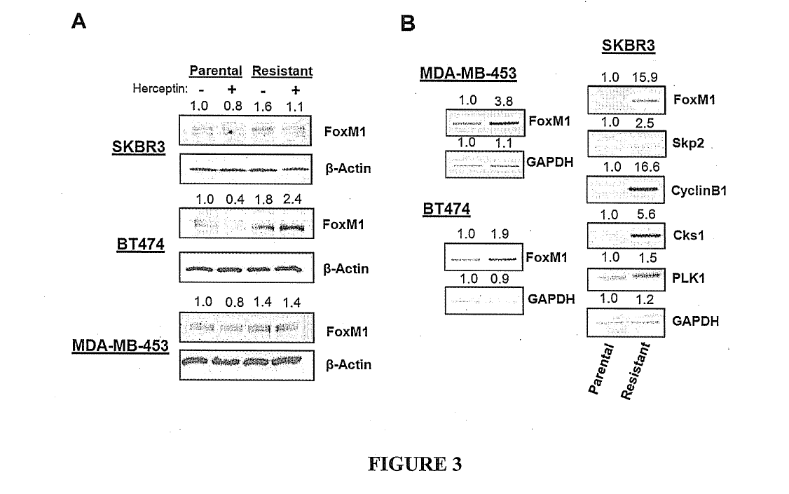

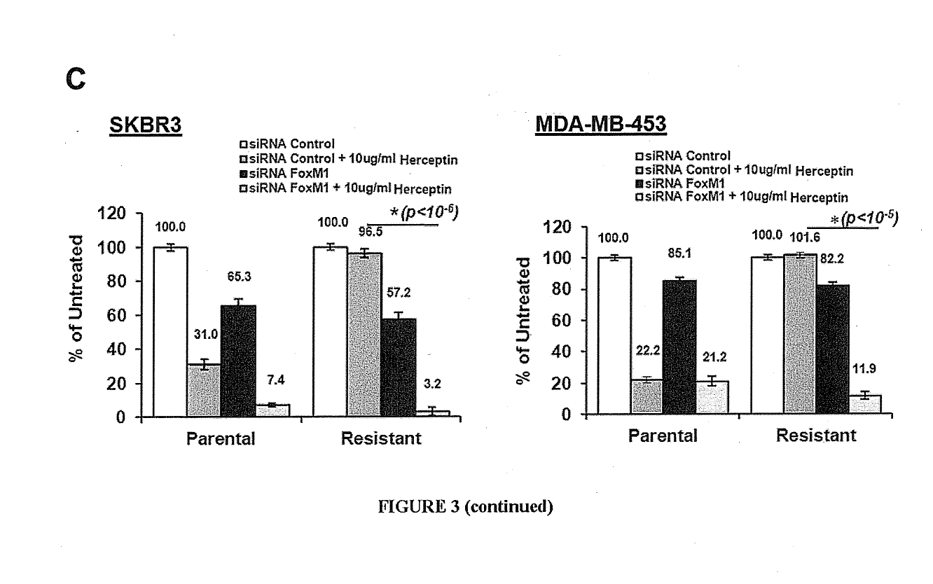

FoxM1 levels were higher in all resistant lines (FIG. 3A). This increase was also reflected at the RNA level (FIG. 3B). To confirm a higher activity of FoxM1, RNA levels of known FoxM1 target genes were assayed by semi-quantitative RT-PCR. RNA was extracted using Trizol (Invitrogen) and cDNA was synthesized using reverse transcriptase (Bio-Rad). Equal amounts of cDNA were used for all PCR reactions (Promega). PCR products were analyzed over a series of cycle numbers in order to ensure that data were produced during the PCR log-scale amplification. Samples were assayed using agarose gel electrophoresis, photographed, and quantified using Image J. The following primers were used:

GAPDH:
(SEQ ID NO: 15)
5′-ACA CCC ACT CCT CCA CCT TT-3′
and
(SEQ ID NO: 16)
5′-TTC CTC TTG TGC TCT TGC TG-3′;
FoxM1:
(SEQ ID NO: 17)
5′-GCA GGC TGC ACT ATC AAC AA-3′
and
(SEQ ID NO: 18)
5′-TCG AAG GCT CCT CAA CCT TA-3′;
CyclinB1:
(SEQ ID NO: 19)
5′-AAA GTC TAC CAC CGA ATC CCT A-3′
and
(SEQ ID NO: 20)
5′-CCA AAA CAC AAA ACC AAA ATG A-3′;
Cks 1:
(SEQ ID NO: 21)
5′-GAA TGG AGG AAT CTT GGC GTT C-3′
and
(SEQ ID NO: 22)
5′-TCT TTG GTT TCTT GGG TAG TGG G-3′;
Polo Like Kinase 1:
(SEQ ID NO: 23)
5′-TGT AGA GGA TGA GGC GTG TTG AG-3′
and
(SEQ ID NO: 24)
5′-AGC AAG TGG GTG GAC TAT TCG G-3′;
Skp2:
(SEQ ID NO: 25)
5′-CAC GAA AAG GGC TGA AAT GTT C-3′
and
(SEQ ID NO: 26)
5′-GGT GTT TGT AAG AGG TGG TAT CGC-3′;
and
stathmin:
(SEQ ID NO: 27)
5′-GCC AGT GTC CTT TAC TTT CCC TCC-3′
and
(SEQ ID NO: 28)
5′-TTC AGT TTC TCC CCT TAG GCC C-3′.

As shown in the SKBR3 resistant line, FoxM1 RNA levels were significantly increased (15-fold) as well as levels of the p27 ubiquitin ligase components Skp2 (2.5-fold) and Cks1 (5.6-fold). Additionally, levels of cell cycle regulators, Polo-like Kinase 1 (1.5-fold) and Cyclin B1 (16.6-fold) were amplified in the resistant line as compared to the parental control line (FIG. 3B). GAPDH is used as a loading control to ensure that the same amount of RNA was added to each reaction. All bands are normalized to GAPDH bands from the same sample and then normalized values from parental and resistant cells can be compared. These results confirmed that increased FoxM1 levels conferred resistance to HERCEPTIN. Experiments were conducted to determine whether targeting FoxM1 could re-sensitize these resistant cells to HERCEPTIN. Knockdown of FoxM1 by siRNA in SKBR3 resistant cells led to a more than 75% percent reduction in cell number when used in conjunction with HERCEPTIN (FIG. 3C, left panel). The control (5′ CAGUCGCGUUUGCGACUGGTT 3′, SEQ ID NO:12) and FoxM1 targeting siRNA (5′ GGACCACUUUCCCUACUUUUU 3′, SEQ ID NO:9) were both from Dharmacon, and purified using standard desalting methods. Prior to transfection, plates are washed and cells are placed in serum-free media. siRNA was added to a final concentration of 7.5 pm to each plate using Lipofectamine 2000 (Invitrogen) transfection. Four hours after transfection, 30% FBS containing media is added to the plates to bring the final concentration to 10%. This effect was also observed in MDA-MB-453 cells (FIG. 3C, right panel). Collectively, these results indicated that FoxM1 was up-regulated in resistant lines and that targeted inhibition of FoxM1 provided a method of sensitizing resistant cells to HERCEPTIN treatment.

### Example 4

### Effects of FoxM1 Overexpression on Resistance to Paclitaxel

It has been previously reported that cells that overexpress HER2/ErbB2 display decreased sensitivity to apoptosis caused by Paclitaxel (Azambuja et al., 2008, “HER2 overexpression/amplification and its interaction with taxane-based therapy in breast cancer,” Ann Oncol 19: 223-32; Yu et al., 1998, “Overexpression of ErbB2 blocks Taxol-induced apoptosis by upregulation of p21Cip1, which inhibits p34Cdc2 kinase,” Mol. Cell 2: 581-91). To determine whether FoxM1 could protect cells from Taxol induced apoptosis, cells overexpressing FoxM1 were treated with Taxol (e.g., Paclitaxel).

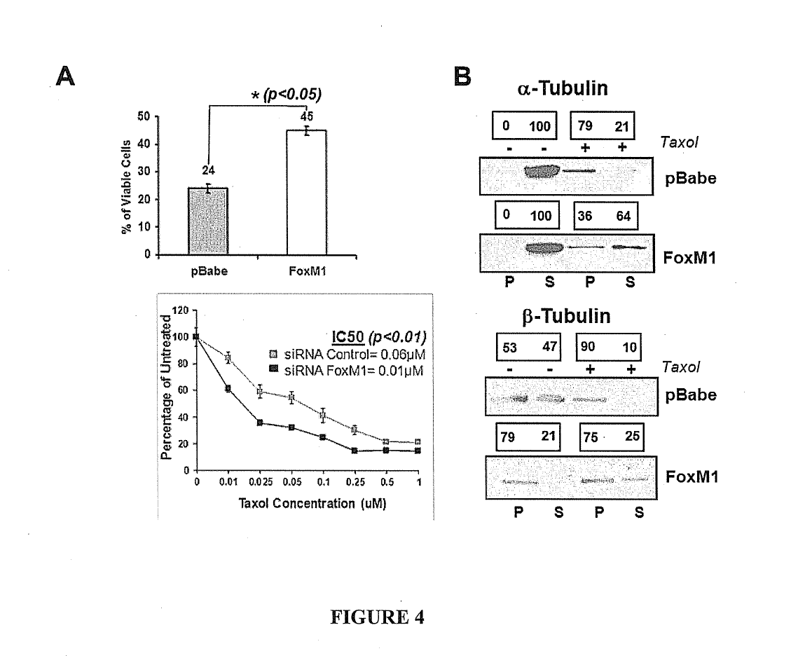

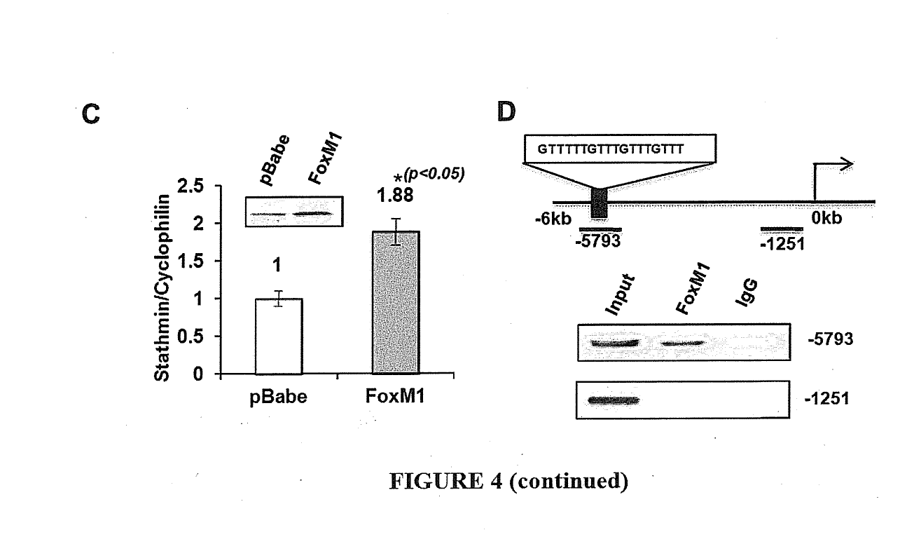

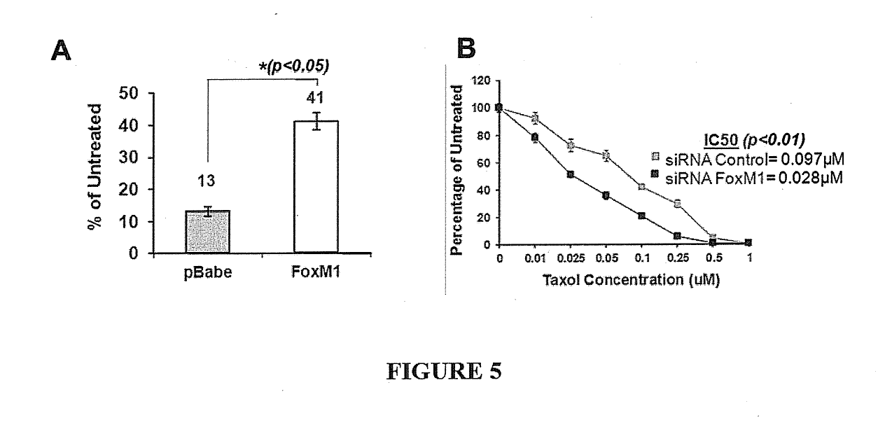

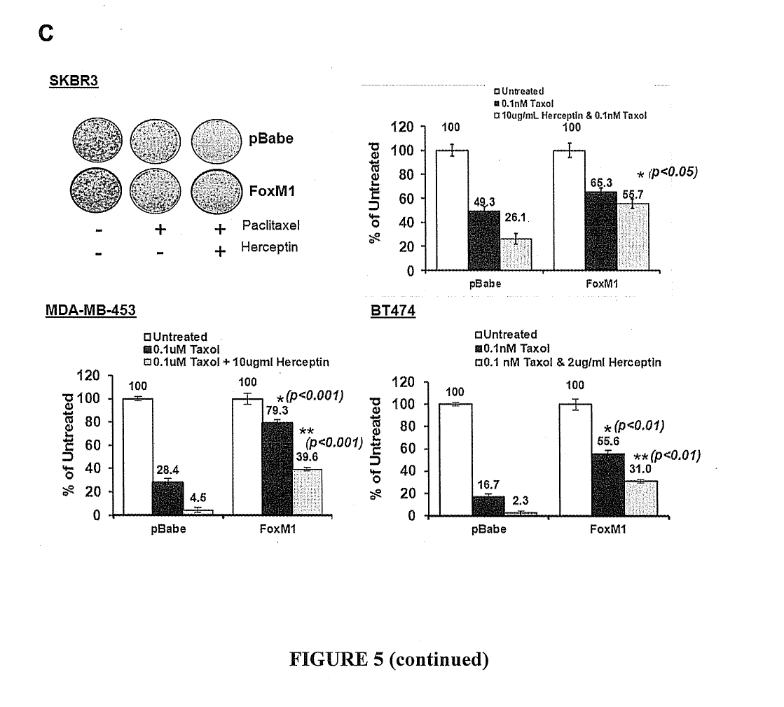

After seven days of treatment in a low dose of paclitaxel (TAXOL) (0.1 μM), only 25% of SKBR3-pBabe cells survived, while nearly 50% of SKBR3-FoxM1 cells survived (FIG. 4A). Cell viability was measured using CellTiter-Glo Luminescent assay (Promega, catalog No. G7570), which measures the amount of oxygenated oxyluciferin directly correlated to the amount of ATP present. This effect was also observed in MDA-MB-453 and BT474 FoxM1 expressing lines (FIG. 5C). Moreover, knockdown of FoxM1 by siRNA in SKBR3 cells sensitized the cells to Taxol treatment as evidenced by a reduced IC₅₀ value in cells transfected with FoxM1-specific siRNA (0.01 uM) as compared to cells transfected with control siRNA (0.06 uM) (FIG. 4A). These results indicate that FoxM1 can protect cells from paclitaxel-induced cell death.

The potential cellular bases by which FoxM1 could prevent paclitaxel induced apoptosis was also investigated. Several mechanisms to counteract paclitaxel-induced apoptosis have been reported, for example, up-regulation of MDR1 (multi-drug resistant protein 1), which is a P-Glycoprotein family member that can shuttle toxins out of cells, up-regulation of the CIAP (inhibitors of apoptosis) family members including survivin, and altered microtubule dynamics (Orr et al., 2003, “Mechanisms of Taxol resistance related to microtubules,” Oncogene 22: 7280-95). No effect of FoxM1 on the levels of MDR1 was detected (data not shown). Also, FoxM1 has been known to positively regulate the CIAP family member survivin and increased expression of survivin has been known to protect cells from Taxol. However, an increased expression of survivin was not observed in the mammary tumor cells assayed herein.

In addition, the possibility that FoxM1 induced altered microtubule dynamics was investigated. Paclitaxel has been known to stabilize tubulin, and thus the ratio of polymerized to soluble microtubule fractions was compared. Cell lysates of SKBR3-pBabe and SKBR3-FoxM1 expressing lines untreated or treated with paclitaxel were fractionated to obtain polymerized and soluble tubulin fractions. Separation of polymerized and soluble fractions was done as previously described (Giannakakou et al., 1997, “Paclitaxel-resistant human ovarian cancer cells have mutant beta-tubulins that exhibit impaired paclitaxel-driven polymerization.,” J Biol Chem 272: 17118-25, incorporated by reference in its entirety herein). Briefly, cells were seeded at 80% confluency in 24-well plates, collected in hypotonic buffer (1 mM MgCl₂, 2 mM EGTA, 0.5% Nonidet P-40, 20 mM Tris-HCl pH 6.8) and centrifuged for 10 minutes at room temperature (14,000 rpm). The supernatant was used as the soluble fraction while the pellet was used as the polymerized fraction. Without treatment, cells showed similar tubulin ratios and nearly all detectable tubulins were in the soluble form (FIG. 4B). Upon treatment with paclitaxel, SKBR3-pBabe cells showed a dramatic shift towards the polymerized fraction for both α- and β-tubulin. Although FoxM1-expressing cells also showed an increase in polymerized fraction for α-tubulin, the ratio of polymerized:soluble α-tubulin was considerably lower in FoxM1-expressing cells as compared with pBabe cells (0.56:1 FoxM1 vs. 3.76:1 pBabe) (FIG. 4B). And the FoxM1-expressing cells did not show a significant change in the ratio for β-tubulin after paclitaxel treatment.

It has been previously established that increased expression and activity of the microtubule destabilizing protein stathmin can confer resistance to paclitaxel-induced apoptosis both in patient samples and cell culture (Balachandran et al., 2003, “Altered levels and regulation of stathmin in paclitaxel-resistant ovarian cancer cells,” Oncogene 22: 7280-05; Alli et al., 2002, “Effect of stathmin on the sensitivity to antimicrotubule drugs in human breast cancer,” Cancer Res 62: 6864-9). The hallmark of increased stathmin activity is a low ratio of polymerized to soluble tubulin as was observed in FoxM1-expressing cells (Giannakakou et al., 1997, “Paclitaxel-resistant human ovarian cancer cells have mutant beta-tubulins that exhibit impaired paclitaxel;—driven polymerization,” J. Biol Chem 272: 17118-25). To investigate this phenomenon in these cells, stathmin RNA expression in pBabe and FoxM1 cell lines was compared. The results showed that the FoxM1-expressing cells expressed 2-fold more stathmin RNA compared to pBabe control cells (FIG. 4C). This difference was also noted at the protein level (FIG. 4C, inset). In addition, chromatin immunoprecipitation (ChIP) of SKBR3 cells was performed as described previously (Park et al, 2009, “FoxM1, a critical regulator of oxidative stress during oncogenesis,” Embo J 28: 2908-18, incorporated by reference in its entirety herein). Briefly, cells were fixed in 1% formaldehyde for 10 minutes to allow crosslinking followed by quenching with 125 nM glycine. Cells were collected and lysed in SDS lysis buffer (1% SDS, 10 mM EDTA, 50 mM Tris pH 8, protease and phosphatase inhibitors). Lysates were sonicated, pre-cleared, and incubated with anti-FoxM1 antibody followed by purification with Protein-A and Protein-G Sepharose beads in the presence of salmon sperm DNA (Upstate). Beads were washed and DNA extracted using a PCR purification kit (Qiagen). The following primers were used for PCR: 5′-CAA ATG TGC TTG CCT TTT AGC C-3′ (SEQ ID NO:29) and 5′-TGG GAT TAC AGA TGT GAG CCA CC-3′ (SEQ ID NO:30) for −5793 and 5′-CAC GGT CAG ACC AAT TTC T-3′ (SEQ ID NO:31) and 5′-TGA TAG GGG AGG AAG AGC AA-3′ (SEQ ID NO:32) as a non-specific control.

ChIP using anti-FoxM1 antibody showed enrichment of the stathmin promoter region, indicating that the observed increases in stathmin RNA and protein levels in FoxM1 expressing lines were likely due to a direct interaction of FoxM1 with the stathmin gene promoter (FIG. 4D). Together, these studies demonstrated that SKBR3-FoxM1 cell lines resistant to paclitaxel-induced apoptosis up-regulated the microtubule destabilizing protein stathmin.

### Example 5

### FoxM1 Overexpression Protects Cells from HERCEPTIN and Paclitaxel in Combination

While the success of HERCEPTIN as a single agent treating breast cancer is significant, the best therapeutic response is seen when HERCEPTIN is used in conjunction with other chemotherapeutic agents such as TAXOL. Therefore experiments were conducted to determine the role of FoxM1 in resistance towards combination therapy.

Pretreatment of both SKBR3-pBabe and FoxM1 cell lines for 72 hours with HERCEPTIN followed by paclitaxel treatment revealed significant differences. FoxM1-expressing cells exhibited resistance to killing by these agents when compared to control pBabe cells. For example, seven days after paclitaxel treatment, only 10-12% of pBabe cells survived, whereas the survival of FoxM1-expressing cells was greater than 40% (FIG. 5A). Knockdown of FoxM1 expression in SKBR3 cells sensitized these cells to combination treatment, as evidenced by a reduction of IC₅₀ value in FoxM1 cells transfected with FoxM1-specific siRNA compared with control siRNA (0.097 uM (siRNA Control) vs. 0.028 uM (siRNA FoxM1)) (FIG. 5B).

The effect of FoxM1 on long-term combination treatment was also investigated by colony forming assays. Cell viability was measured using CellTiter-Glo Luminescent assay (Promega), which measured the amount of oxygenated oxyluciferin having a direct correlation to ATP present. For colony forming assays, 3-5×10³ cells were plated in triplicate in a 24-well plate, and 24 hours later were treated with 10 ug/ml of HERCEPTIN for 72 hours followed by 0.1 μM Taxol treatment for 4 hours. The cells were maintained in HERCEPTIN thereafter. After 17 days cells were fixed and stained with crystal violet. Quantification was done using Adobe Photoshop. All p-values were calculated using Student's t-test.

Quantification of colony numbers showed that approximately 55% of FoxM1-expressing SKBR3 cells survived after combination therapy, whereas only 26% of pBabe lines survived the treatment (FIG. 5C). The ability of FoxM1 to mediate resistance to combination therapy was observed also in a comparison of pBabe vs. FoxM1-expressing MDA-MB-453 (4.5 vs. 39.6%) and BT474 (2.3 vs. 31%) cell lines (FIG. 5C). These results clearly indicated that FoxM1 can protect breast cancer cells from treatment with HERCEPTIN and Paclitaxel in combination.

### Example 6

### An ARF-Derived Peptide Inhibitor of FoxM1 Sensitizes Mammary Tumor Cells to HERCEPTIN Treatment

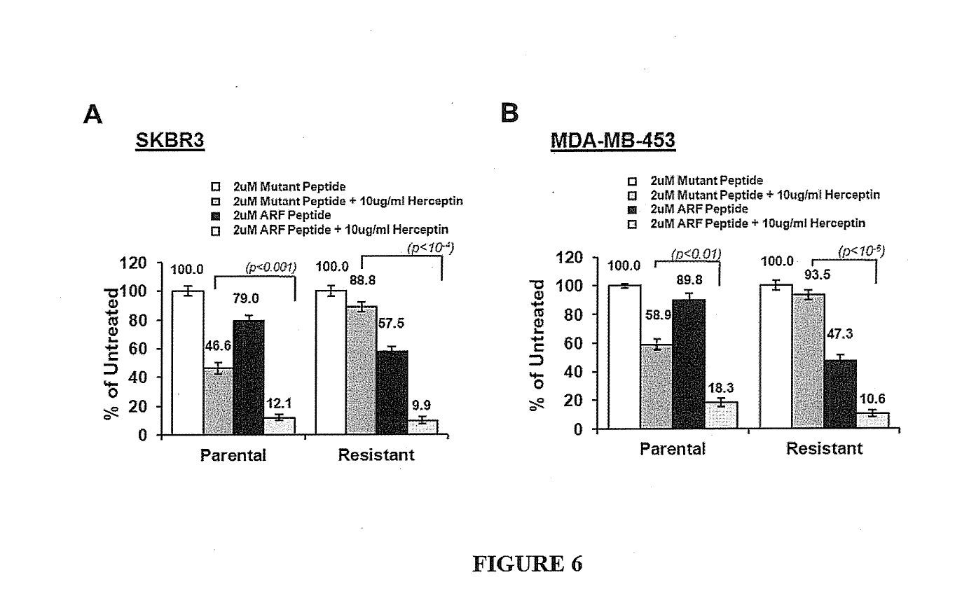

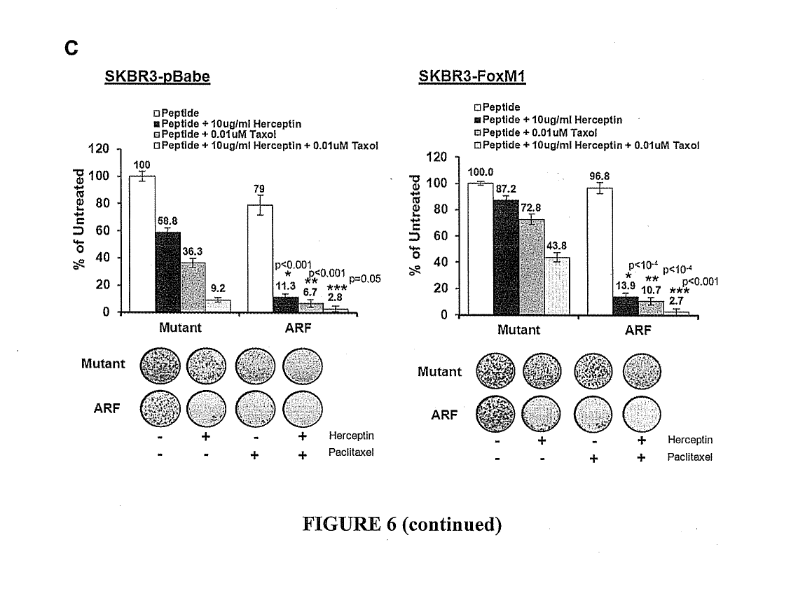

Studies have shown that FoxM1 is inhibited by a small peptide that contains a 19-amino acid region of the p19ARF protein (residues 26 to 44) (SEQ ID NO:2). This peptide has been shown to reduce proliferation and induce apoptosis of hepatocellular carcinoma cells in vivo (see, U.S. Pat. Nos. 7,635,673 and 7,799,896, which are incorporated herein by reference in their entireties; see also, Gusarova et al., 2007, “A cell-penetrating ARF peptide inhibitor of FoxM1 in mouse hepatocellular carcinoma treatment,” I. Clin, Invest 117:99-111). Treatment with the ARF-derived peptide and trastuzumab led to a 90% reduction in cell numbers in both SKBR3 and MDA-MB-453 resistant cells as measured by colony forming assays (following the same protocol as described above in Example 5) (FIG. 6A). Similar results were seen in parental lines treated with the same peptide and trastuzumab (FIG. 6A). Treatment of resistant cells with a mutant peptide (SEQ ID NO:79) did not change colony number compared to parental lines receiving the same mutant peptide and therefore was used as a control.

The ability of the ARF-peptide to sensitize FoxM1-expressing cells to treatment was also investigated. Addition of the ARF-peptide to HERCEPTIN, paclitaxel, or combination treatment showed a dramatic reduction in cell number compared to mutant peptide. The ARF peptide sensitized pBabe cells to all treatments, resulting in greater cell killing at the same dosage as compared to the mutant peptide (FIG. 6C). Most notably, addition of the ARF-peptide resulted in more than 97% cell killing in FoxM1-expressing cells, i.e., resulted in less than 3% of FoxM1-expressing cells surviving the combination treatment. The data suggested that the addition of the ARF peptides can provide chemotherapeutic and clinical benefits for breast cancer treatment with HERCEPTIN, paclitaxel, or combinations thereof.

### Example 7

### Characterization of FoxM1 Expression in Breast Cancer and Mammary Development

### Animal Model

All animal experiments were preapproved by the UIC institutional animal care and use committee. WAP-rtTA-Cre mice were obtained from the Mouse Repository of the National Cancer Institute (NCI, Frederick, Md.). FoxM1 FL/FL mice have been previously characterized (Wang et al., 2005, “Forkhead box M1 regulates the transcriptional network of genes essential for mitotic progression and genes encoding the SCF (Skp2-Cks1) ubiquitin ligase,” Mol Cell Biol 25, 10875-94). C57BL/6 mice were purchased from Charles River Laboratories (Wilmington, Mass.). For deletion studies, mice were given 2 mg/mL of doxycycline (Sigma) dissolved in 5% sucrose (Sigma) solution in water bottles.

### Tumor Grade Analysis

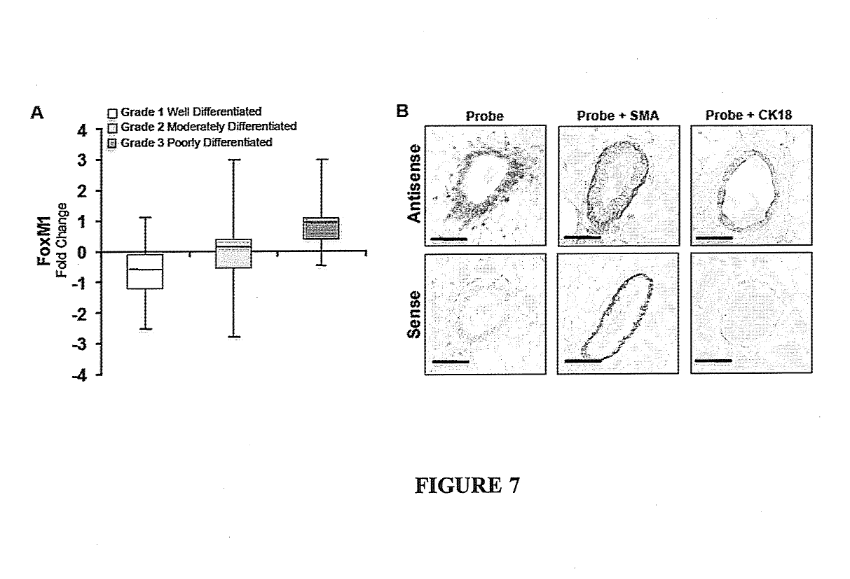

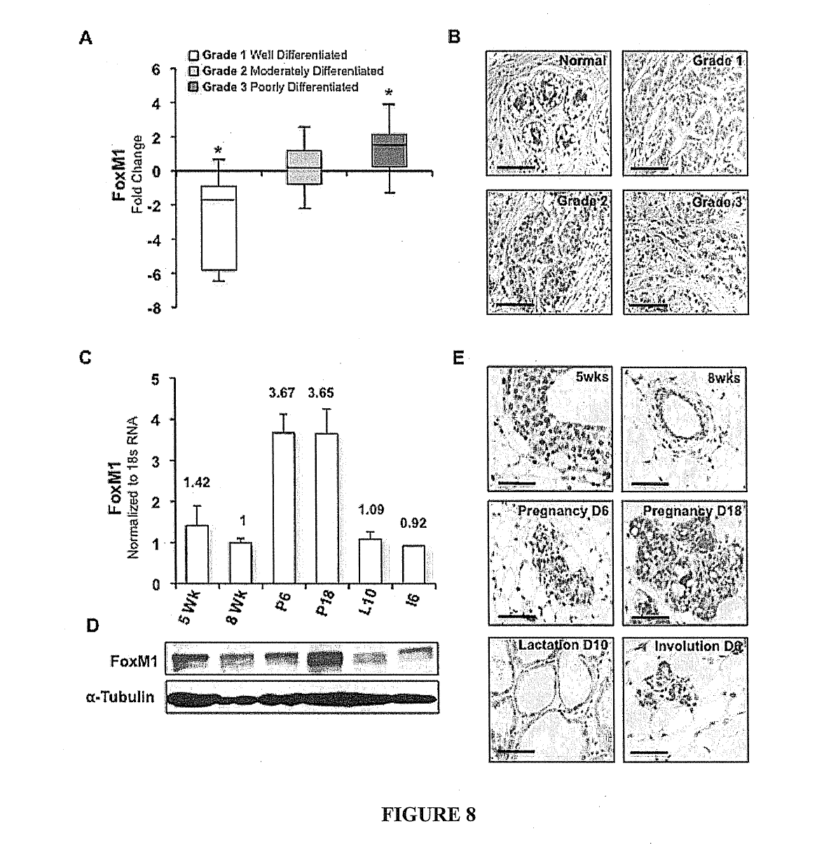

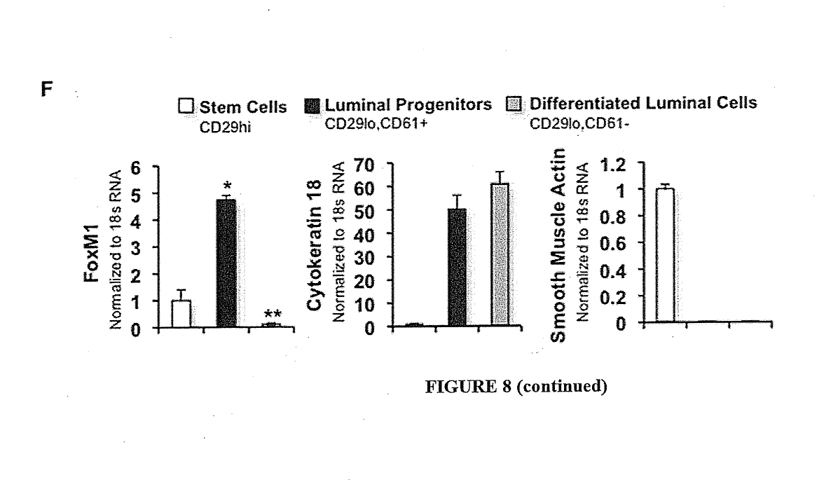

Analysis of publicly available microarray data (Oncomine, Compendia Bioscience, Ann Arbor, Mich.) demonstrated that FoxM1 expression increased with tumor grade in human breast cancers (see FIG. 7A and FIG. 8A, similar results obtained from different datasets). Breast cancer datasets were exported from Oncomine to analyze expression of FoxM1 and GATA-3 in human tumor arrays, which were scored by two independent pathologists. All p-values were calculated using Student's t-test. This pattern was further validated using tissue arrays that allow for analysis of expression and localization. While levels of FoxM1 were faint and cytoplasmic in normal tissue as well as grade 1 tumors, staining intensity increased and became primarily nuclear in grade 3 tumors, confirming that FoxM1 expression was inversely correlated with tumor differentiation (FIG. 8B).

To investigate the role of FoxM1 in regulating mammary differentiation, the normal expression pattern throughout key stages of postnatal mammary development was examined using quantitative RT-PCR and western blot analysis. RNA was extracted with Trizol (Invitrogen) and cDNA was synthesized by reverse transcriptase (Bio-Rad). cDNA was amplified using SYBR Green mastermix (Bio-Rad) and analyzed via iCycler software and the delta-delta Cₜ method. Data from mouse studies was normalized to 18S RNA and from human studies to GAPDH. All primer sequences are shown in Table 1 below. For western blot analysis, tissue protein extracts were homogenized in lysis buffer containing: 50 mM Hepes-KOH, 300 mM NaCl, 1 mM EDTA, 1 mM EGTA, 1 mM DTT, 0.1% Tween 20, and 10% glycerol. Extracts from cell lines were prepared in lysis buffer containing: 1 mM EDTA, 0.15M NaCl, 0.05M Tris-HCl pH 7.5, and 0.5% Triton-X. Phosphatate Inhibitor Cocktail Set II (Calbiochem) and protease inhibitor (Roche) were added to lysis buffers before each experiment.

FoxM1 was detected at the RNA (FIG. 8C) and protein (FIG. 8D) levels during puberty (5 weeks), adulthood (8 weeks), pregnancy (days 6 and 18), lactation, and involution in mice. FoxM1 expression levels varied considerably: pregnancy, a period of ductal growth and expansion showed highest levels of expression, while involution, characterized by apoptosis and remodeling, exhibited the lowest expression. This pattern was also reflected at the protein level by immunohistochemistry (FIG. 8E). For immunohistochemistry, glands were fixed in modified Davidson's fixative (30% of 37% formaldehyde-15% ethanol-5% acetic acid) for 48 hours, rinsed in PBS, left in 10% PBS-buffered formalin overnight and embedded in paraffin. For staining, antigen retrieval was done using sodium citrate buffer (10 mM sodium citrate, pH 6.0 and 0.05% Tween) and anti-FoxM1 antibodies (Santa Cruz Biotechnology, K-19) were incubated overnight at a 1:50 dilution. Visualization was done using an avidin-biotin conjugate (ABC) and 3,3′-diaminobenzidine (DAB) and counterstained using Hematoxylin (Polyscientific).

Mammary terminal end buds are present during puberty in the mouse (5-6 weeks of age). This structure is of particular significance because the cap cells or those found in the invading front make up the progenitor cell population (Williams and Daniel, 1983, “Mammary ductal elongation: differentiation of myoepithelium and basal lamina during branching morphogenesis,” Dev Biol 97:274-90; Smalley and Ashworth, 2003, “Stem cells and breast cancer: A field in transit,” Nat Rev Cancer 3:832-44). Strong nuclear staining for FoxM1 was observed in cap and progenitor cells (FIG. 8E, top left). At all stages of development FoxM1 expression was primarily found in cells of luminal lineage.

To confirm this observation, in situ hybridization was employed to identify FoxM1 mRNA followed by immunostaining for luminal and myoepithelial cell types. For in situ hybridization, 322 bp mouse FoxM1 probes were amplified from cDNA using the following primers: 5′-GCTATCCAACTCCTGGGAAGATTC-3′ sense (SEQ ID NO:33) and 5′-CAATGTCTCCTTGATGGGGGTC-3′ antisense (SEQ ID NO:34). T7 polymerase (Ambion) and digoxigenin (DIG)-labeled nucleotides (Roche) were used to make labeled RNA probes. Labeling of paraffin-embedded sections was performed using the IsHyb in situ hybridization kit (Biochain). Sections were counterstained in nuclear fast red (Vector Labs) or fixed briefly in paraformaldehyde and stained using antibodies to smooth muscle actin or cytokeratin 18 as indicated.

The results of these experiments showed a clear overlap of FoxM1 antisense probe hybridization and cytokeratin 18 immunostaining, indicating that FoxM1 was expressed mainly in luminal cells (FIG. 7B). The timing and pattern of expression suggested that FoxM1 levels were higher in cells that were less differentiated. Previously defined flow cytometry markers were used to separate mammary stem cells (CD29hi), luminal progenitors (CD291o, CD61+), and differentiated luminal cells (CD291o, CD61−) (Stingl et al., 2006, “Purification and unique properties of mammary epithelial stem cells,” Nature 439:993-7; Shackleton et al., 2006, “Generation of a functional mammary gland from a single stem cell,” Nature 439:84-8; Asselin-Labat et al., 2007, “Gata-3 is an essential regulator of mammary-gland morphogenesis and luminal-cell differentiation,” Nat Cell Biol 9:201-9). These cell types were sorted from 8-week old C57BL/6 mice. Total RNA from sorted cells was analyzed for FoxM1 expression using quantitative RT-PCR as described above. The level of FoxM1 in stem cells was ten-fold higher than differentiated cells while luminal progenitors showed a nearly 50-fold increase (FIG. 8F). Expression of cytokeratin 18 and smooth muscle actin (SMA) were used to determine the purity of luminal and myoepithelial populations respectively (FIG. 8F). Taken together, these results demonstrated that FoxM1 expression is highest in luminal progenitor cells and decreased upon differentiation.

TABLE 1
Primers Information

| | Sense (5′→3′) | Antisense (5′→3′) |
|---|---|---|
| Gene Name | [SEQ ID NO] | [SEQ ID NO] |
| FoxM1 | GAGGAAAGAGCACCTTCAGC | AGGCAATGTCTCCTTGATGG |
| | [35] | [36] |
| GATA-3 | CCGAAACCGGAAGATGTCTA | AGATGTGGCTCAGGGATGAC |
| | [37] | [38] |
| 18s RNA | ACATCGACCTCACCAAGAGG | TCCCATCCTTCACATCCTTC |
| | [39] | [40] |
| Rb1 | TGATAACCTTGAACCTGCTTG | GGCTGCTTGTGTCTCTGTATTTGC |
| | TCC | [42] |
| | [41] | |
| Estrogen | AAGGCGGCATACGGAAAGAC | ATCCAACAAGGCACTGACCATC |
| Receptor | [43] | [44] |
| α | | |
| Amphi- | ACTCACAGCGAGGATGACAA | TAACGATGCCGATGCCAATAG |
| regulin | GG | [46] |
| | [45] | |
| Cytokeratin | TTCAGTCTCAACGATGCCCTG | ATTAGTCTCGGACACCACTCTGCC |
| 18 | G | [48] |
| | [47] | |
| Smooth | ATCATTGCCCCTCCAGAACG | GCTTCGTCGTATTCCTGTTTGC |
| Muscle | [49] | [50] |
| Actin | | |
| Cadherin | AATGTGCCTGAGAGGTCCAAT | CGAGAAATAGGGTTGTCCTTCAAG |
| 11 | G | [52] |
| | [51] | |
| Human | GCAGGCTGCACTATCAACAA | TCGAAGGCTCCTCAACCTTA |
| FoxM1 | [53] | [54] |
| Human | TGTCAGACCACCACAACCAGA | TGGATGCCTTCCTTCTTCATAGTC |
| GATA-3 | C | [56] |
| | [55] | |
| Human | ACACCCACTCCTCCACCTTT | TTCCTCTTGTGCTCTTGCTG |
| GAPDH | [57] | [58] |

Human GATA-3 Methylation Specific PCR

| | | |
|---|---|---|
| Set 1 (Site | TTATCGGTGGGATAGTTTGC | AACCGCTAACCCGAAAATAC |
| −1431) | [59] | [60] |
| Set 2 (Site | CTTGTAATAGTTGAAGCGTGTT | ATACCTTTAACTAAAACGTC |
| −747) | T | [62] |
| | [61] | |
| Beta- | TGGTGATGGACGAGGTTTAGTA | AACCAATAAAACCTACTCCTCCCT |
| Actin | AGT | TA |
| | [63] | [64] |

Mouse GATA-3 ChIP

| | | |
|---|---|---|
| Site −1686 | CTGACGCTGTTCGTTCTGGAGA | AAGATTTGCCTCCGAACC |
| | [65] | [66] |
| Site −721 | ACGCCTCCTCCTCCTCCTCTAC | AGCACACCTCCGACAGCCAG |
| | [67] | [68] |
| Site −291 | GTCACACTCGGATTCCTCTCTC | CCCCAAAAAAAAGCAGCAGACAC |
| | C | [70] |
| | [69] | |

Human GATA-3 ChIP

| | | |
|---|---|---|
| Site −1730 | CAAGTGGGCTCAGGAGAAA | GTGTGAGGGTCGTCGTGTT |
| | [71] | [72] |
| Site −1431 | TTCAGAACTTACTTTCAGGGAC | AATGCTGCCAGGAGAGGGAGTG |
| | GG | [74] |
| | [73] | |
| Site −747 | TCTCATCCCTCACTGTTGCCAC | TGTCATTGTCACCTCTTTCCCG |
| | [75] | [76] |
| Non- | TTTTACGGGGCAACTACGGC | CAGTGGCATCCATTAGCAGGTC |
| Specific | [77] | [78] |

### Example 8

### Acute Loss of FoxM1 Results in Expansion of Differentiated Luminal Cells

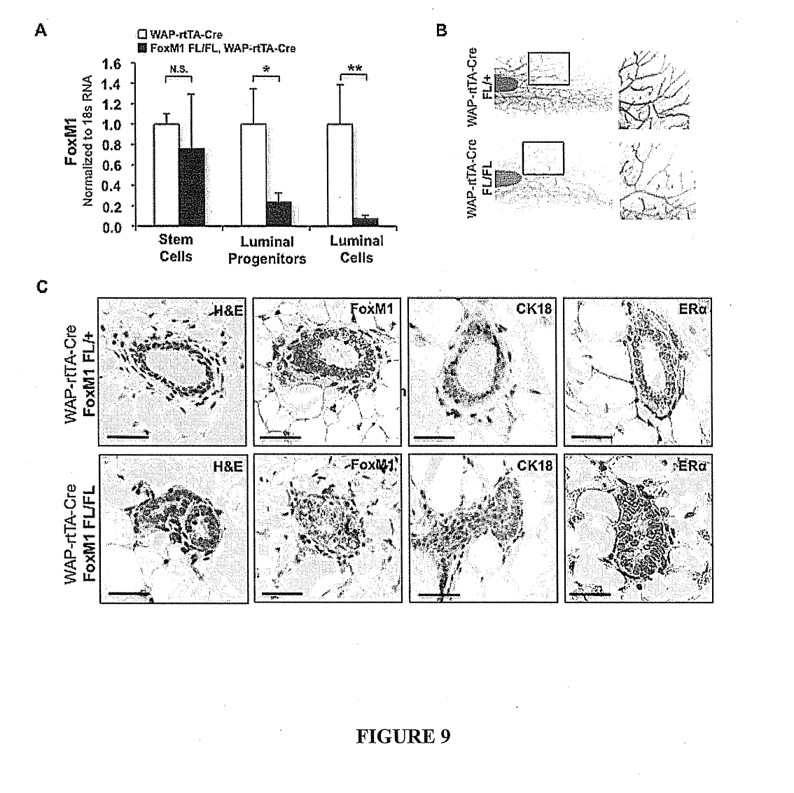

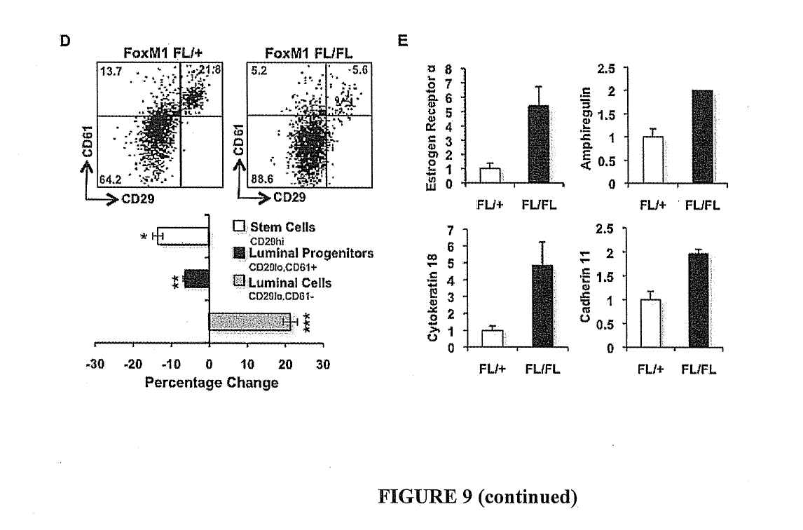

FoxM1 deletion in mammary tissue in transgenic mice was analyzed to determine if endogenous FoxM1 regulates luminal cell differentiation. Transgenic mice harboring mammary-specific doxycycline-inducible Cre construct (WAP-rtTA-Cre) were crossed with transgenic mice harboring the FoxM1 gene flanked by LoxP sites (FoxM1 FL/FL). The FoxM1 FL/+ and FoxM1 FL/FL littermates, expressing the inducible Cre, were given doxycycline in their drinking water for 5 or 15 days. After 5 days of treatment, mammary glands were sorted into stem cells, luminal progenitors, and differentiated luminal cells to determine the pattern of FoxM1 deletion. An 80% reduction of FoxM1 expression in luminal progenitors and 90% in differentiated luminal cells was observed while stem cells did not show a significant reduction (FIG. 9A). This pattern was similar to previous reports using the WAP promoter to drive Cre expression for gene knockout studies (Jiang et al., 2010, “Rb deletion in mouse mammary progenitors induces luminal-B or basal-like/EMT tumor subtypes depending on p53 status,” J Clin Invest 120: 3296-309).

Following 5 days of treatment with doxycycline, FoxM1 protein was still detectable by immunohistochemistry. However, after 15 days of doxycycline administration, FoxM1 protein was no longer detectable by immunostaining (FIG. 9C). Thereafter, mammary glands were removed for carmine alum whole mount staining by spreading the gland on glass slides and placed in Carnoy's fixative (60% ethanol, 30% chloroform and 10% glacial acetic acid) overnight. Glands were hydrated in an alcohol gradient and left in carmine alum (Sigma) overnight then cleared in xylene. For green fluorescent protein (GFP) imaging, glands were removed, spread on a glass slide, fixed in 4% paraformaldehyde overnight, cleared in 50% glycerol in PBS for 4 hours, then 75% glycerol for 4 hours, and then 100% glycerol overnight. Glands were imaged using a fluorescent dissecting microscope. Whole-mount staining using carmine alum showed that FoxM1 FL/FL, WAP-rtTA-Cre mice had sparse and narrow ductal branching while FoxM1 FL/+ appeared identical to wildtype mice (FIG. 9B). Wildtype and WAP-rtTA-Cre expressing mice showed structures and staining patterns indistinguishable from FoxM1 FL/+ mice, indicating an absence of Cre toxicity and that FoxM1 FL/+ mice were valid controls. On closer examination of recombinant glands by sectioning, FoxM1 FL/FL WAP-rtTA-Cre mice showed a loss of FoxM1, confirming that the gene was deleted, while FL/+ mice showed FoxM1 staining that mirrored the normal gland. FoxM1 FL/FL mice exhibited abnormal histological staining by H&E. Unlike in normal mammaries, glands from FoxM1 FL/FL WAP-rtTA-Cre mice were not composed of a single layer of epithelial cells and the lumens were filled with cells that expanded beyond the myoepithelial layer. Staining of cytokeratin 18 and estrogen receptor alpha indicated that these cells were differentiated luminal epithelium, suggesting an expansion of the differentiated pool (FIG. 9C).

Stem, progenitor, and differentiated pools were analyzed after 15 days of treatment to examine the effects of FoxM1 deletion on mammary cell subtypes. There was found an approximate 20% increase in the percentage of differentiated luminal cells in these pools with a concomitant loss in stem and progenitor populations demonstrating that loss of FoxM1 in mammary gland resulted in a shift towards the differentiated state (FIG. 9D). Consistent with that observation, deletion of FoxM1 resulted in an increase in markers of luminal differentiation, including estrogen receptor alpha, amphiregulin, cytokeratin 18, and cadherin 11 (FIG. 9E). Taken together, these data demonstrated that loss of FoxM1 in the adult gland led to an increase in differentiated cells and a loss of progenitor pools.

In other experiments, mouse mammary gland was regenerated with elevated levels of FoxM1 to examine the consequences of high levels of FoxM1 on mammary differentiation. Primary mammary epithelial cells were used to generate mammosphere cultures as previously described (Dontu et al., 2003, “In vitro propagation and transcriptional profiling of human mammary stem/progenitor cells,” Genes Dev 17:1253-70, incorporated in its entirety by reference herein). Specifically, the No. 4 inguinal mammary glands were removed from 6-8 week old C57BL/6 mice. Glands were digested for 6 hours in collagenase/hyaluronidase, cells collected by centrifugation, red blood cells lysed using a 0.8% ammonium chloride solution, and glands further digested using 0.25% trypsin (Cellgro) and dispase. DNaseI (Sigma, 10 ug/ml) was used to remove DNA from dead cells. Cells were suspended in Hanks' balanced salt solution and 2% FBS and filtered through 0.4 uM strainer (BD Biosciences). Cells were counted and incubated with retrovirus as described below. All reagents were from Stem Cell Technologies unless otherwise noted.

The plasmid construct pMigR-FoxM1-EGFP was generated by cloning FoxM1 cDNA into the pMigR-EGFP plasmid (Luk Van Parijis et al, 1999, Immunity 11:281). Cells were plated at 40% confluency and infected with retroviral constructs using lipofectamine2000 (Invitrogen). After 24 hours, media were changed to 3% FBS and DMEM and fresh virus was used to infect mammospheres. DMEM with low FBS concentration at 3% was used to minimize the FBS that stem cells were exposed to. Fresh virus in the volume of 2 ml was added to mammosphere cells from above along with 10 ug/ml polybrene. Cells were incubated with virus at 37° C. for 120 minutes and gently mixed every 20 minutes. After 2 hours, cells were centrifuged, supernatant was removed, and cells were resuspended in media containing DMEM/F12 (Invitrogen/Gibco), serum-free B27 (Gibco), 20 ng/mL EGF (Peprotech), 20 ng/ml FGF (Peprotech), 4 μg/mL Heparin (Sigma), and Penicillin/Streptamycin (Cellgro, 100U of penicillin, 100 ug of Streptamycin). Cells were plated at a density of 5×10⁵/75 cm² flask. Spheres were allowed to form for 7 days.

At the end of 7 days spheres were collected, digested in 0.05% trypsin for 10 minutes at 37° C., resuspended in Hanks' balanced salt solution and 2% FBS, centrifuged, and suspended in fresh media at a concentration of 1×10⁶/ml. GFP, dsRed (red fluorescent protein), or double positive cells were sorted using Beckman Coulter MoFlo sorter and Summit software. One thousand sorted cells were resuspended in matrigel (BD Biosciences) and were implanted into the cleared mammary fat pad of 3-4 week old C57BL/6 mice as previously described (DeOme et al., 1959, “Development of mammary tumors from hyperplastic alveolar nodules transplanted into gland-free mammary fat pads of female C3H mice,” Cancer Res 19:515-20, incorporated in its entirety by reference herein). All data are shown normalized to the control gland from the same animal. All analyses were performed after 7-8 weeks of regrowth.

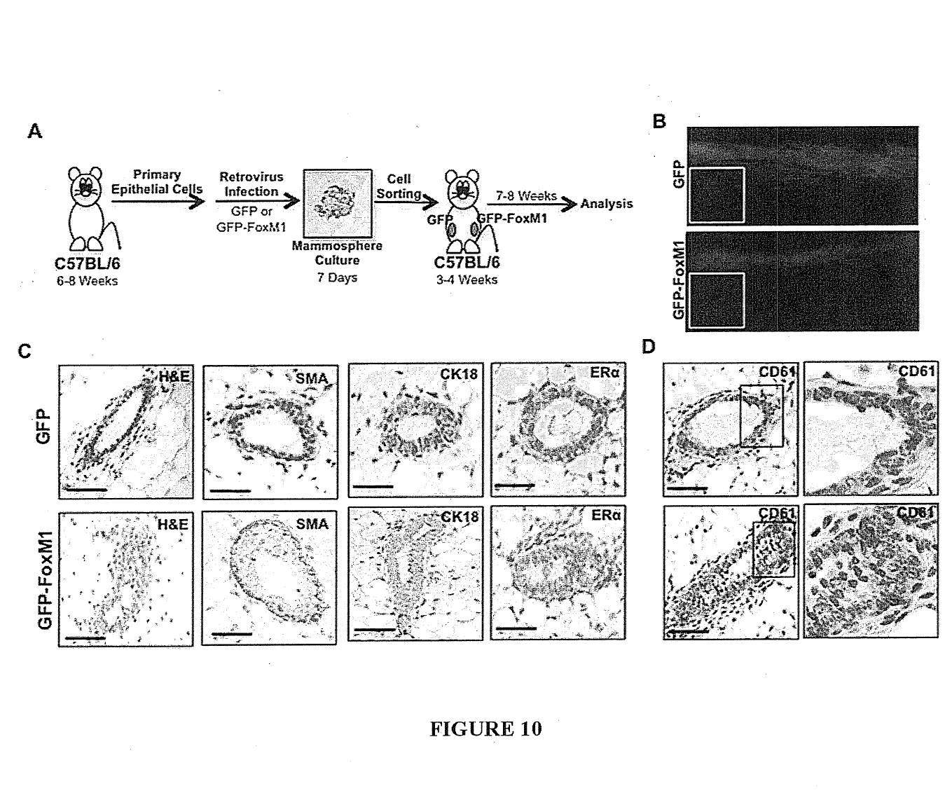

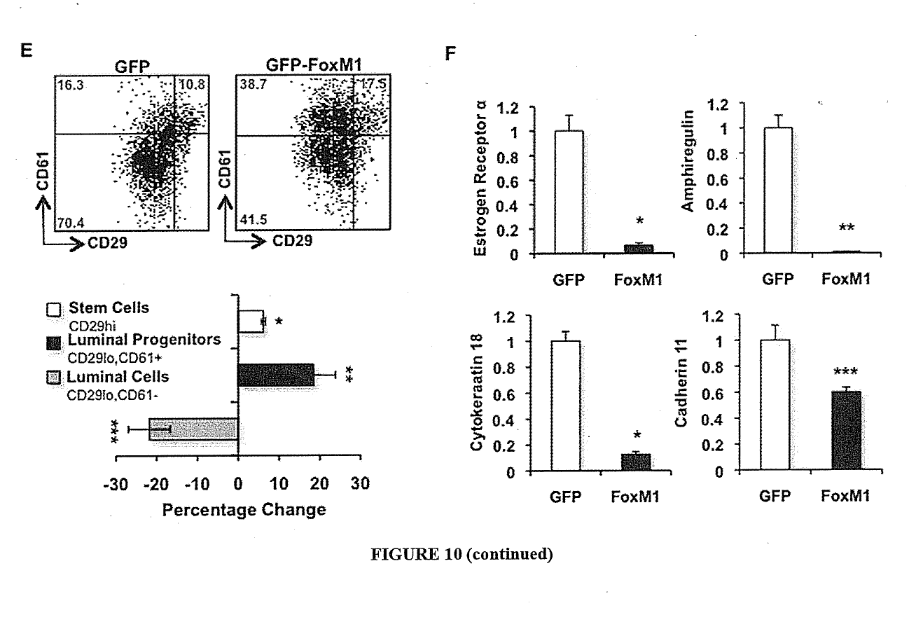

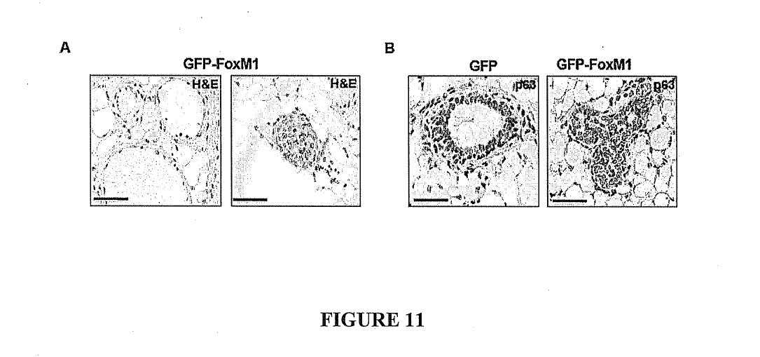

GFP-positive mammosphere cells were identified by sorting and injected into the cleared fat pads of 3-4 week old mice. GFP and GFP-FoxM1 positive cells were placed on contralateral sides of the same animal, allowing each animal to function as their own control (FIG. 10A). Addition of retrovirus or GFP did not have an effect on mammary development as glands expressing GFP mirrored those of wildtype mice. Carmine alum whole mount staining and GFP staining and imaging were done as described above. On whole mount analysis, GFP-FoxM1 glands showed a considerable narrowing in comparison to their GFP counterparts (FIG. 10B). Regenerated glands were sectioned and stained to analyze the architecture of individual ducts. GFP glands showed the expected staining pattern, a single layer of epithelial cells surrounded by myoepithelial cells. GFP-FoxM1 expressing glands showed two distinct phenotypes within the same gland by H&E staining: hyperplastic features and an “empty lumen.” The “empty lumen” was observed less often and was made up of a region where basal cells were present but luminal cells were absent. Hyperplastic regions showed excessive cell infiltration, which led to distorted lumen architecture, with epithelial cells filling the lumen or spreading beyond the basal layer (FIG. 10C and FIG. 11A).

To further investigate the altered architecture of FoxM1-expressing glands, sections were stained with markers of myoepithelial and luminal cell lineages. Staining with the basal marker, smooth muscle actin (SMA), revealed that GFP glands, as expected, showed a ring of SMA positive cells surrounding the lumen, FoxM1 expressing glands, however, showed SMA-positive cells surrounded by luminal cells (FIG. 10C). This phenotype was previously observed in glands expressing shRNA to Cbf-1 (a notch cofactor) and was correlated with an expansion of undifferentiated mammary cells (Bouras et al., 2008, “Notch signaling regulates mammary stem cell function and luminal cell-fate commitment,” Cell Stem Cell 3:429-41). These cells did not stain positive with the basal marker p63, indicating that they were not misplaced myoepithelial cells (FIG. 11B).

Cytokeratin 18 staining shows a uniform luminal restricted staining pattern (Hennighausen, et al., 2005, “Information networks in the mammary gland,” Nat Rev Mol Cell Biol 6:715-25). GFP glands exhibited this typical staining pattern, while FoxM1 glands showed a punctate pattern distinct from differentiated luminal cells (FIG. 10C). The expanded cells did not stain positive for estrogen receptor alpha, indicating an expansion of an undifferentiated cell of luminal origin (FIG. 10C). These results were supported by staining for CD61, a marker of luminal progenitors: glands expressing FoxM1 exhibited an increased number and intensity of CD61 positive cells as compared to control glands (FIG. 10D).

To confirm expansion of an undifferentiated cell type in FoxM1-expressing glands, cell populations were analyzed using flow cytometry. For cell cycle analysis by flow cytometry, cells were trypsinized, pelleted, and resuspended in propidium iodide (PI) solution (50 ug/ml PI, 0.1 mg/ml RNaseA, 0.05% Triton-X; all reagents were purchased from Sigma). After 40 minutes of incubation at 37° C., cells were analyzed using a flow cytometer. Glands were processed using sequential enzyme digestion, blocked using an antibody to CD16/CD32 and hematopoietic stem cells were removed using an epithelial cell enrichment kit (Stem Cell Technologies). Cells were stained using CD24-PE (BD Biosciences), CD29-APC (e-Biosciences), CD61-biotin and streptavidin PE-Cy7 (BD Biosciences). Mammary gland comprising two retroviruses (GFP- and dsRed-expressing) were stained using CD24-PE-Cy7 (BD Biosciences), CD29-APC, and CD61-biotin and streptavidin pacific blue (BD Biosciences). Analysis was done using a Beckman-Coulter flow cytometer and Summit software.

Comparing FoxM1 to paired GFP controls showed a distinct shift away from the differentiated state. The luminal progenitor pool expanded considerably, nearly 20%, with a similar reduction in the percentage of differentiated cells, suggesting that addition of FoxM1 resulted in a failure of cells to properly exit the luminal progenitor pool and differentiate fully (FIG. 10E). Consistent with this notion, RT-PCR data showed a reduction in estrogen receptor alpha, amphiregulin, cytokeratin 18, and cadherin 11, markers of luminal differentiation (FIG. 10F).

### Example 10

### FoxM1 is a Negative Regulator of GATA-3 In Vivo

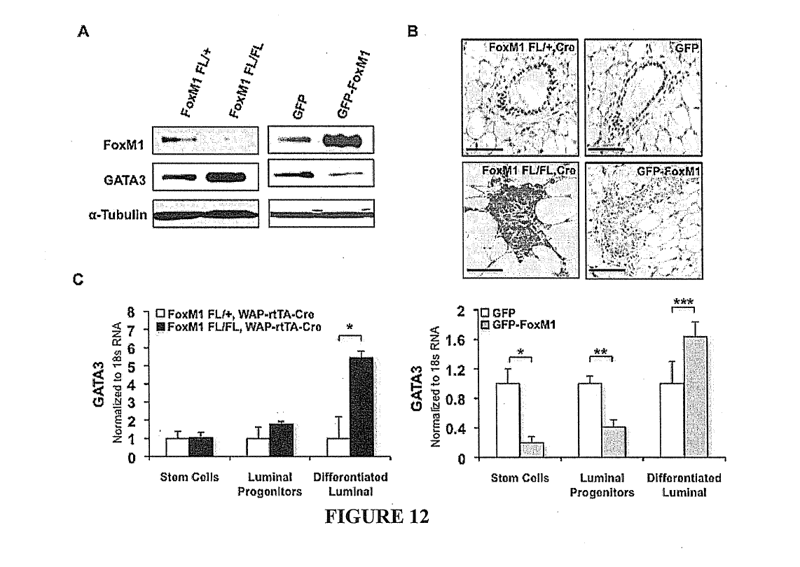

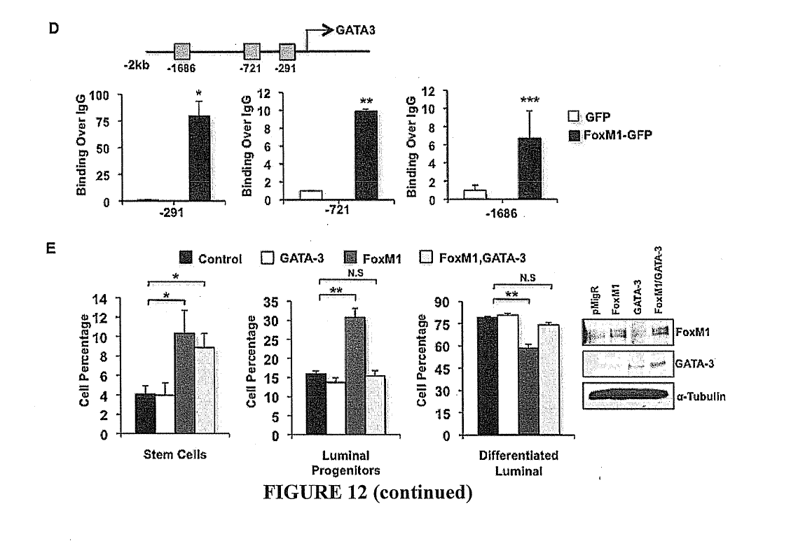

GATA-3 is considered as a master regulator of mammary differentiation. GATA-3 expression in both FoxM1 deletion and over-expression transgenic mouse models was analyzed to investigate if FoxM1 functions as a negative regulator of GATA 3. Protein extracts from mammary tissue were homogenized in lysis buffer containing: 50 mM Hepes-KOH, 300 mM NaCl, 1 mM EDTA, 1 mM EGTA, 1 mM DTT, 0.1% Tween 20, and 10% glycerol. Extracts from cell lines were prepared in lysis buffer containing: 1 mM EDTA, 0.15M NaCl, 0.05M Tris-HCl pH 7.5, and 0.5% Triton-X. Phosphatate Inhibitor Cocktail Set II (Calbiochem) and protease inhibitor (Roche) were added to the lysis buffers before each experiment; all reagents are from Sigma-Aldrich unless otherwise noted. Glands in which FoxM1 was deleted showed a considerable increase in GATA-3 protein levels by western blot analysis. Conversely, GATA-3 protein levels were significantly decreased in GFP-FoxM1 expressing glands compared to their GFP counterparts (FIG. 12A). Immunohistochemical staining also reflected the decrease in protein levels (FIG. 12B). GATA-3 generally showed a pattern of strong nuclear staining in luminal cells and that was evident in control glands (FIG. 12B). FoxM1 deletion resulted in increased staining intensity while over-expression resulted in decreased and diffuse staining pattern for GATA-3 (FIG. 12B)

GATA-3 RNA expression in sorted populations from glands from FoxM1 deleted and over-expressing transgenic mice was analyzed. RNA was extracted with Trizol (Invitrogen) and cDNA was synthesized by reverse transcriptase (Bio-Rad). cDNA was synthesized and amplified as described above. Data from mouse studies were normalized to 18S RNA and from human studies to GAPDH. All primer sequences are shown in Table 1. After Cre-mediated deletion of FoxM1, a five-fold increase in GATA-3 mRNA was observed in differentiated cells. Stem cells did not show any change, which was expected given that FoxM1 was not deleted in that population. Additionally, there was a slight (but not significant) increase in GATA-3 in the luminal progenitors (FIG. 12C). FoxM1 expression in the over-expression transgenic mouse model exhibited increase of FoxM1 in all cell types. Accordingly, glands expressing FoxM1 displayed a significant reduction in GATA-3 in stem and luminal progenitors, while differentiated cells showed higher expression of GATA-3 (FIG. 12C). This unexpected finding in differentiated cells could be attributed to the possibility that when FoxM1 was upregulated, an elevated expression of GATA-3 was required for the cells to maintain the differentiated state.

The mouse GATA-3 promoter contains three FoxM1 consensus sequences within 2 kb of the transcriptional start site. Whether FoxM1 directly regulated GATA-3 at the RNA level was investigated using chromatin immunoprecipitation (ChIP) assay. Cells were fixed in 1% formaldehyde for 10 minutes to allow crosslinking and then quenched with 125 nM glycine. For in vivo ChIP assays, single cell suspensions were generated using collagenase/hyaluronidase followed by fixing. Cells were collected and lysed in SDS lysis buffer (1% SDS, 10 mM EDTA, 50 mM Tris pH 8, protease and phosphatase inhibitors). Lysate was sonicated, pre-cleared, and incubated with antibodies against GFP (Clontech, JL-8), GATA-3 (Santa Cruz HG3-31), FoxM1 (Major et al., 2004, “Forkhead box M1B transcriptional activity requires binding of Cdk-cyclin complexes for phosphorylation-dependent recruitment of p300/CBP coactivators” Mol Cell Biol 24: 2649-61), DNMT3b (Imgenex 52A1018), or Rb (Cell Signaling, 4H1) followed by purification with Protein-A and Protein-G Sepharose beads in the presence of salmon sperm DNA (Upstate). Beads were washed and DNA extracted using a PCR purification kit (Qiagen). PCR products were visualized by gel electrophoresis or analyzed using SYBR Green (Bio-Rad), normalized to the IgG control (Santa Cruz Biotechnology). PCR primer sequences are provided in Table 1.

In vivo chromatin immunoprecipitation assay showed that FoxM1 bound to all of these sites in the regenerated mouse mammary gland (FIG. 12D). Taken together, the data indicated that FoxM1 can bind and repress transcription of GATA-3 in mice in vivo.

To determine whether inhibition of mammary luminal differentiation by FoxM1 was linked to repression of GATA-3, GATA-3 was coexpressed with FoxM1 using retroviruses in mammary stem cells. The plasmid construct pMigR-FoxM1-EGFP was generated by cloning FoxM1 cDNA into pMigR-EGFP (Luk Van Parijs et al., supra). The pMigR-dsRed plasmid construct was made by substituting EGFP with dsRed (Clontech) in pMigR, and the GATA-3-dsRed construct was made by cloning PCR amplified GATA-3 cDNA into pMigR-dsRed. After sorting for expression, these cells were used to regenerate mammary epithelium as described schematically in FIG. 10A. Reconstituted glands were harvested and cell populations analyzed by FACS analysis. Coexpression of GATA-3 reversed the defects observed in FoxM1-expressing mammary glands. Sorting of glands into stem cells, luminal progenitors, and differentiated cells indicated a significant reversal of the FoxM1 phenotype by coexpression of GATA-3 (FIG. 12E). These observations suggested that repression of GATA-3 was involved in FoxM1 inhibition of mammary progenitor differentiation.

### Example 11

### FoxM1 Promotes GATA-3 Methylation in an Rb-Dependent Manner

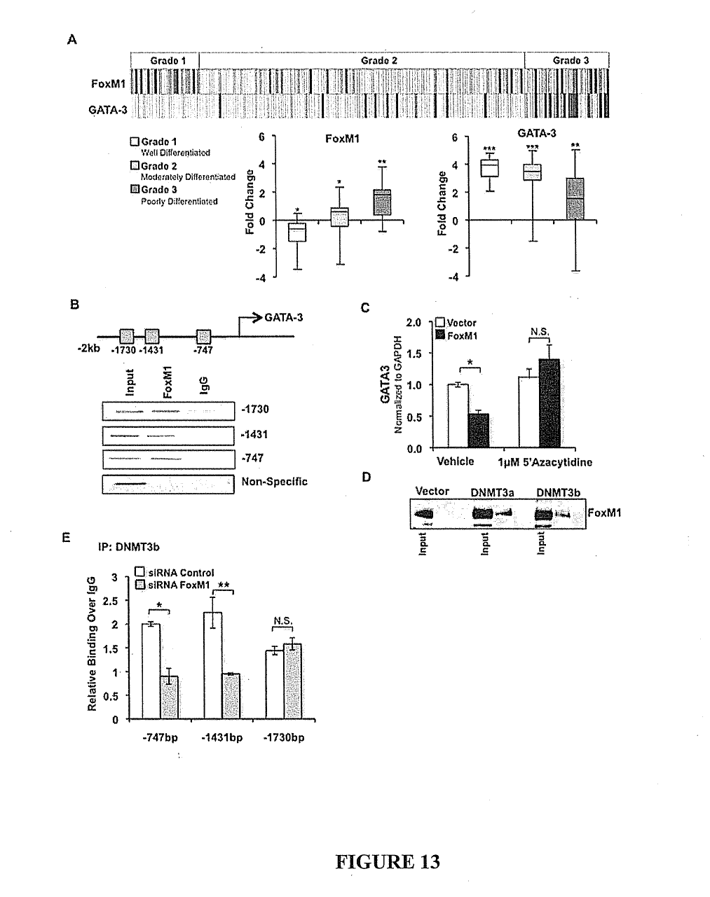

The results set forth in Example 10, showing that FoxM1 inhibits GATA-3, suggested an inverse correlation between GATA-3 and FoxM1 expression in breast tumor samples. Analyses of publicly available database for FoxM1 and GATA-3 expression patterns in human samples were consistent this expectation (FIG. 13A). In addition, direct binding of FoxM1 to human GATA-3 promoter was confirmed. Bioinformatic analysis identified three putative binding sites for FoxM1 in the 2 kb upstream of the transcriptional start site. Chromatin immunoprecipitation assay (ChIP) (performed under the same protocol described in example 11) showed that FoxM1 bound to all three of these sites and not to a non-specific control sequence, indicating that FoxM1 could regulate GATA-3 transcriptional levels in human breast cancer cells (FIG. 13B).

Previous studies showed that the promoter of GATA-3 could be targeted for DNA methylation during tumor progression (Yan et al., 2000, “CpG island arrays: an application toward deciphering epigenetic signatures of breast cancer,” Clin Cancer Res 6:1432-8). To test if GATA-3 repression by FoxM1 was methylation dependent, FoxM1 binding to and inhibition of GATA-3 was measured in the presence of the methyltransferase inhibitor, 5′ azacytidine (5′AZA). Addition of 5′AZA ablated repression of GATA-3 by FoxM1 in the human breast cancer cell line MDA-MB-453, demonstrating that repression is methylation dependent (FIG. 13C). In mammalian systems, methylation patterns are generated and maintained by the DNA methyltransferase family of proteins including, DNMT1, DNMT3a, and DNMT3b (Jones and Baylin, 2002, “The fundamental role of epigenetic events in cancer,” Nat Rev Genet 3:415-28). DNMT1 is responsible for replication-associated methylation, while DNMT3a and 3b are considered to be “de novo” methylators, responsible for dynamic changes in cellular methylation patterns Immunoprecipitation experiments demonstrated that FoxM1 bound to both DNMT3a and DNMT3b (FIG. 13D).

DNMT3b has been specifically implicated in mammary tumor biology. It was shown to be responsible for the hypermethylated phenotype in mammary tumors and decreased expression of tumor suppressor genes (Girault et al., 2003, “Expression analysis of DNA methyltransferases 1, 3A, and 3B in sporadic breast carcinomas,” Clin Cancer Res 9: 4415-22; Roll et al., 2008, “DNMT3b overexpression contributes to a hypermethylator phenotype in human breast cancer cell lines,” Mol Cancer 7:15). The possibility that FoxM1 could function in a complex with DNMT3b and target the GATA-3 promoter for methylation was investigated. ChIP assay was performed as described in previous examples using an antibody specific to DNMT3b. Cells were treated with either siRNA to FoxM1 or control siRNA. In the presence of control siRNA, DNMT3b bound to regions of the GATA-3 promoter that contain FoxM1 binding sites. The binding was significantly decreased when cells were treated with siRNA to FoxM1, indicating that DNMT3b binds to the GATA-3 promoter at −747 and −1431 in a FoxM1 dependent manner (FIG. 13E).

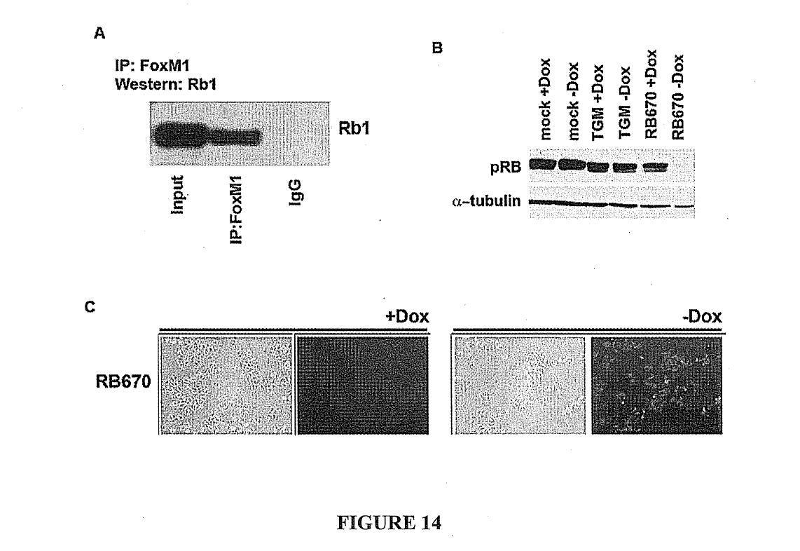

Previous studies indicated that the tumor suppressor Rb can bind to FoxM1 (Major et al., 2004, “Forkhead box M1B transcriptional activity requires binding of Cdk-cyclin complexes for phosphorylation-dependent recruitment of p300/CBP coactivators,” Mol Cell Biol 24:2649-61; Wierstra et al., 2006, “Transcription factor FOXM1c is repressed by RB and activated by cyclin D1/Cdk4,” Biol Chem 387:949-6) and the binding was confirmed by the current studies (FIG. 14A). Whether FoxM1 requires Rb for repressing GATA-3 transcription was investigated using a doxycycline-inducible shRNA system in MCF7 cells to knockdown Rb expression (FIGS. 14B and 14C). To produce the inducible knockdown system, MCF7 cells were first infected with viral particles carrying the pRetroX-Tet-off Advanced vector (Clontech) to establish constitutive expression of the tetracycline-controlled transactivator, tTa-Advanced. Cells with stably integrated constructs were selected by using G418 sulfate for two weeks. Isogenic clones were isolated by plating the cells in limiting dilutions on 10 cm plates, and tTA-Advanced expression was validated by RT-qPCR. Inducibility was assessed by infecting tTA-Advanced positive cells with retroviral particles comprising the pRetroX-Tight-Pur-Luc construct that expresses a tTA-inducible luciferase reporter. Infection continued for three days and Luciferase assay was performed using the Luciferase Dual Reporter Assay System (Promega, catalog No. E1910). Clones showing the highest tTA-Advanced expression and luciferase inducibility were used to produce second stable lines. In all, ˜10 clones were isolated per line, all of which showed at least some expression of tTA-Advanced. The clone showing greater than 20-fold inducibility by luciferase assays was used to produce the second stable lines.

The second stable cell lines carrying vector for expressing miR-30-based shRNA specific to Rb, or the empty control vector TGM, were made by infecting tTA-Advanced expressing clones with TMP-RB.670¹ retroviral particles (“RB670”), or control retroviral particles, and selecting under puromycin dihydrochloride for several days for cells harboring integrated constructs. Individual clones were generated by limiting dilutions on 10 cm plates and validated by performing induction assays for 6 days. In particular, clones were evaluated for inducible GFP expression via fluorescent microscopy as well as western blot analysis for pRB protein level.

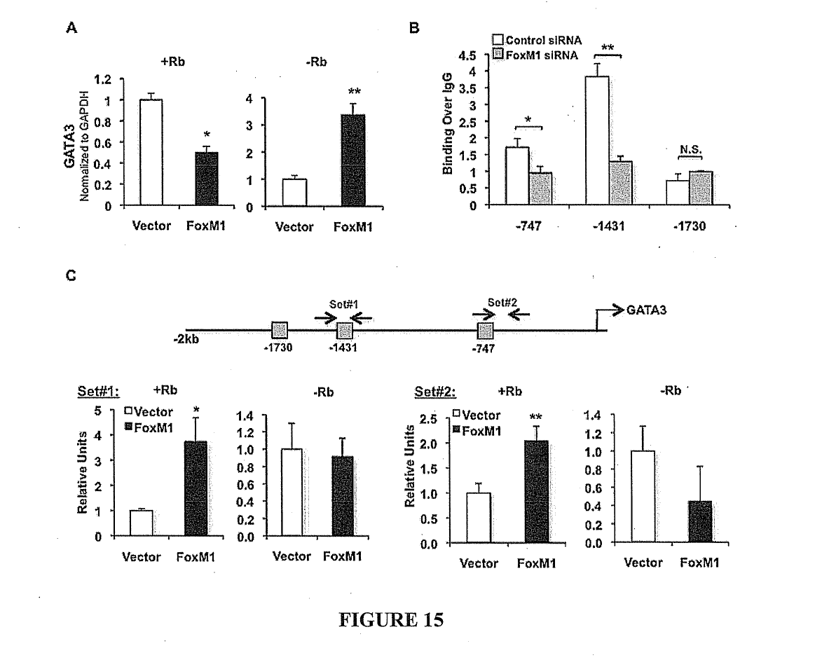

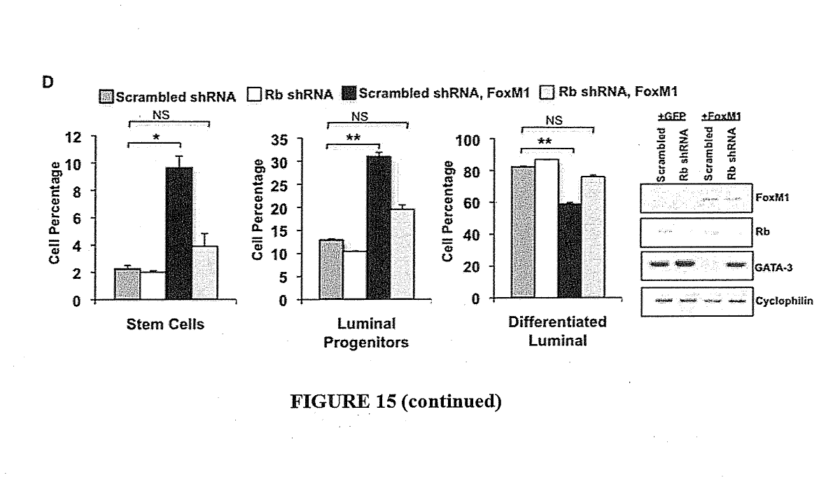

In the absence of Rb, addition of FoxM1 failed to repress GATA-3 and in fact led to a considerable increase of GATA3 expression (FIG. 15A). Additionally, ChIP experiments as previously described in previous examples were conducted using control siRNA or siRNA specific to FoxM1 to show that Rb binding to GATA-3 promoter was FoxM1 dependent (FIG. 15B). The ChIP data showed that Rb could not bind to the GATA-3 promoter in the absence of FoxM1 (FIG. 15B).

The methylation status of the GATA-3 promoter using methylation-specific PCR was studied. Genomic DNA was isolated using Perfect Pure DNA isolation kit (5 Prime). Bisulfite conversion for determining methylation was performed using EZ DNA Methylation kit (Zymo Research). Conversion efficiency was determined to be greater than 95% using primers to converted and unconverted beta actin. Bisulfite-converted DNA was amplified using methylation-specific PCR as described (Herman et al., 1996, “Methylation-specific PCR: a novel PCR assay for methylation status of CpG islands,” Proc Natl Acad Sci USA 93: 9821-6; Liu et al., 2009, “The 14-3-3sigma gene promoter is methylated in both human melanocytes and melanoma,” BMC Cancer 9:162, each of which are incorporated by reference in their entireties herein). Those primers (SEQ ID NOs:59-64) did not amplify non-converted DNA but did amplify SssI methylase treated, bisulfate-converted DNA. Expression of FoxM1 led to a considerable increase in methylation of GATA-3 compared to control transfection. This increase was ablated in the absence of Rb (FIG. 15C), demonstrating that the methylation and subsequent repression of GATA-3 was Rb-dependent. Mouse mammary glands expressing scrambled shRNA or RB-specific shRNAs (SEQ ID NOs:80-83), either in the presence or absence of FoxM1, were generated to study whether knockdown of Rb in vivo ablated FoxM1-mediated inhibition of differentiation. The No. 4 inguinal mammary glands were removed from 6-8 week old C57BL/6 mice. Glands were digested for 6 hours in collagenase/hyaluronidase. Cells were collected by centrifugation, red blood cells lysed using a 0.8% ammonium chloride solution, and glands further digested using 0.25% trypsin (Cellgro) and dispase. DNaseI (Sigma) was used to remove DNA from dead cells. Cells were suspended in Hanks' balanced salt solution and 2% FBS and filtered through 0.4 uM strainer (BD Biosciences). Cells were counted and incubated in retrovirus as described below. All reagents are from Stem Cell Technologies unless otherwise noted.

As disclosed above, the pMigR-FoxM1-EGFP plasmid construct was generated by cloning FoxM1 cDNA into pMigR-EGFP. pMigR-dsRed was made by replacing EGFP in pMigR with dsRed expression construct (Clontech) and GATA-3-dsRed was made by cloning the PCR amplified GATA-3 cDNA into pMigR-dsRed. Scrambled and shRNA constructs against Rb1 were purchased from Origene. Retrovirus was generated using 293 Ampho packaging cell line. Cells were plated at 40% confluency and transfected with retroviral constructs using lipofectamine2000 (Invitrogen). After 24 hours, media was changed to 3% FBS and DMEM and fresh virus was used to infect mammospheres. Low DMEM was used to minimize the FBS that stem cells are exposed to. 2 ml of fresh virus was added to mammosphere cells from above along with 10 ug/ml polybrene. Cells were incubated with virus at 37° C. for 120 minutes and gently mixed every 20 minutes. After 2 hours, cells were centrifuged, supernatant was removed, and cells were resuspended in media containing DMEM/F12 (Invitrogen/Gibco), serum-free B27 (Gibco), 20 ng/mL EGF (Peprotech), 20 ng/ml FGF (Peprotech), 4 μg/mL Heparin (Sigma), and Penicillin/Streptamycin (Cellgro). Cells were plated at a density of 5×10⁵/75 cm² flask. Spheres were allowed to form for 7 days.

At the end of 7 days spheres were collected, digested in 0.05% trypsin for 10 minutes at 37° C., resuspended in Hanks' balanced salt solution and 2% FBS, centrifuged, and suspended in fresh media at a concentration of 1×10⁶/ml. GFP, dsRed, or double positive cells were sorted using Beckman Coulter MoFlo sorter and Summit software. One thousand sorted cells were resuspended in matrigel (BD Biosciences) and were implanted into the cleared mammary fat pad of 3-4 week old C57BL/6 mice as previously described (DeOme 1959, supra). All data were normalized to the control gland from the same animal. All analysis was performed after 7-8 weeks of regrowth.

The cell sorting experiments demonstrated that expression of FoxM1 led to an inhibition of differentiation that was alleviated by the knockdown of Rb (FIG. 15D). Taken together, the data suggested that FoxM1 functions in a complex with DNMT3b and Rb to inhibit GATA-3 expression and mammary luminal differentiation.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1-96. (canceled)

97. A pharmaceutical composition for inhibiting tumor growth comprising a combination of a FoxM1 inhibitor and trastuzumab or paclitaxel or both trastuzumab and paclitaxel, wherein the combination is in a therapeutically effective amount, and a pharmaceutically acceptable excipient, diluent or carrier.

98. The pharmaceutical composition of claim 97 wherein the combination comprises a FoxM1 inhibitor and trastuzumab and paclitaxel.

99. The pharmaceutical composition of claim 97 wherein the FoxM1 inhibitor comprises an inhibitory P19ARF peptide.

100. The pharmaceutical composition of claim 99 wherein the inhibitory P19ARF peptide comprises a peptide having the sequence of SEQ ID NO:6 or SEQ ID NO:7.

101. The pharmaceutical composition of claim 97 wherein the FoxM1 inhibitor comprises a FoxM1-specific siRNA.

102. The pharmaceutical composition of claim 101, wherein the FoxM1-specific siRNA comprises a polynucleotide having the sequence of SEQ ID NO:8, SEQ ID NO:9, SEQ ID NO:10, or SEQ ID NO:11.

103. The pharmaceutical composition of claim 97 wherein the FoxM1 inhibitor comprises a thiazole antibiotic.

104. The pharmaceutical composition of claim 103, wherein the thiazole antibiotic is siomycin A or thiostrepton.

105. The pharmaceutical composition of claim 97 wherein the FoxM1 inhibitor is an antioxidant.

106. The pharmaceutical composition of claim 105 wherein the antioxidant is N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

107. A method of treating cancer in a patient comprising administering to a patient in need thereof a combination of a FoxM1 inhibitor and trastuzumab or paclitaxel or both trastuzumab and paclitaxel, wherein the combination effectively inhibits cancer, and wherein the cancer is ovarian cancer, breast cancer, small cell lung cancer, non-small cell lung cancer, colorectal cancer, malignant peripheral nerve sheath tumors, cervical cancer, leukemia, prostate, Kaposi's sarcoma, metastatic melanoma, pancreatic cancer, head and neck tumors, meningiomas, basal cell carcinoma, and gliomas.

108. The method of claim 107, wherein the FoxM1 inhibitor comprises an inhibitory P19ARF peptide.

109. The method of claim 108, wherein the inhibitory P19ARF peptide comprises a peptide having the sequence of SEQ ID NO:6 or SEQ ID NO:7.

110. The method of claim 107, wherein the FoxM1 inhibitor comprises a FoxM1-specific siRNA.

111. The method of claim 110, wherein the FoxM1-specific siRNA comprises a polynucleotide having the sequence of SEQ ID NO:8, SEQ ID NO:9, SEQ ID NO:10, or SEQ ID NO:11.

112. The method of claim 107 wherein the FoxM1 inhibitor comprises a thiazole antibiotic.

113. The method of claim 112, wherein the thiazole antibiotic is siomycin A or thiostrepton.

114. The method of claim 107, wherein the FoxM1 inhibitor comprises an antioxidant.

115. The method of claim 114, wherein the antioxidant is N-acetyl-L-cysteine (NAC), catalase, 4-Hydroxy-2,2,6,6-tetramethylpiperidine-1-oxyl (Tempol), or manganese(III)-5,10,15,20-tetrakis(N-methylpyridinium-2-yl)porphyrin pentachloride (MnTM-2-PyP).

116. A method of claim 107 further comprising identifying and treating trastuzumab-resistant cancer in a patient comprising the steps of
(a) obtaining a breast cancer tissue sample from a patient in need of the treatment, wherein the breast cancer tissue sample is HER2/ErbB2 positive;
(b) detecting FoxM1 expression in the breast cancer tissue sample using a reagent that specifically detects FoxM1; and
(c) administering to the patient a FoxM1 inhibitor and trastuzumab or paclitaxel or both trastuzumab and paclitaxel when FoxM1 expression is detected in the breast cancer tissue sample.

117. The method of claim 116, wherein FoxM1 expression is detected in the nucleus of the cells of the breast cancer tissue sample.

118. The method of claim 116, further comprising the steps of obtaining a control breast tissue sample and assaying the control breast tissue sample to detect FoxM1 expression therein, wherein in step (c) a FoxM1 inhibitor is administered to the patient with trastuzumab or paclitaxel or both trastuzumab and paclitaxel if FoxM1 expression is greater in the breast cancer tissue sample than in the control breast tissue sample.

119. The method of claim 107, further comprising identifying and treating paclitaxel-resistant cancer in a patient comprising the steps of
(a) obtaining a cancer tissue sample from a patient in need of the treatment;
(b) detecting FoxM1 expression in the cancer tissue sample using a reagent that specifically detects FoxM1, wherein detecting FoxM1 expression in the cancer tissue sample indicates that the cancer is resistant to paclitaxel treatment;
(c) obtaining a control tissue sample; and
(d) assaying the control tissue sample to detect FoxM1 expression therein,
wherein a FoxM1 inhibitor is administered to the patient with paclitaxel if FoxM1 expression in the cancer tissue sample is greater than FoxM1 expression in the control tissue sample.

120. The method of claim 119, wherein the FoxM1 expression is detected in the nucleus of the cells of the cancer tissue sample.

121. The method of claim 119, wherein the reagent comprises one or more FoxM1-specific primers, and the level of FoxM1 expression is determined by reverse-transcriptase polymerase chain reaction (RT-PCR).

122. The method of claim 119, wherein the reagent is a FoxM1 specific antibody and the level of FoxM1 expression is determined by an immunoassay.
